(19) United States
(12) Patent Application Publication
(10) Pub. No.: US 2024/0366806 A1
(43) Pub. Date: Nov. 7, 2024

(54) ADOPTIVE IMMUNOTHERAPY COMPOSITIONS AND METHODS OF TRACKING

(71) Applicant: CASE WESTERN RESERVE UNIVERSITY, Cleveland, OH (US)

(72) Inventors: David Wald, University Heights, OH (US); Agata Exner, Westlake, OH (US)

(21) Appl. No.: 18/575,937

(22) PCT Filed: Jul. 5, 2022
(86) PCT No.: PCT/US2022/036120

§ 371 Date: Jan. 2, 2024

Related U.S. Application Data
(60) Provisional application No. 63/217,834, filed on Jul. 2, 2021.

(51) Int. Cl. A61K 49/22; A61K 35/17; A61K 39/00

(52) U.S. Cl. CPC A61K 49/223; A61K 35/17; A61K 39/4611; A61K 39/4613; A61K 39/4631

(57) ABSTRACT

An adoptive immunotherapy composition includes an enriched population of immune cells, wherein at least about 10%, at least about 20%, at least about 30%, at least about 40%, at least about 50%, at least about 60%, at least about 70%, or more immune cells include intracellular nanobubbles.

### RELATED APPLICATION

This application claims priority from U.S. Provisional Application No. 63/217,834 filed Jul. 2, 2021, the subject matter of which is incorporated herein by reference in its entirety.

### BACKGROUND

Despite advances, most patients with relapsed/refractory malignancies lack adequate treatment options. Recently T-cell and Natural Killer (NK) cell therapies have shown promise in at least subsets of cancer patients. Despite the promise of T-cell and NK cell therapies, the trafficking and survival of the infused cells in humans is almost completely unknown (outside of the blood). As this fundamental knowledge is necessary to identify ideal tumor types/patients for cellular therapies and to optimize dosing, the development of non-invasive methodologies to track these cells is important. Current approaches to solve this problem is limited by potential safety issues including radioactive exposure and/or heavy ion toxicity, and difficulty in serial tracking due to complex instrumentation and/or the requirement for repetitive radiation exposure.

### SUMMARY

Embodiments described herein relate to an adoptive immunotherapy composition that includes an enriched population of immune cells that are labeled with nanoscale ultrasound contrast agent or nanobubbles (NBs) and to the use of the adoptive immunotherapy composition in treating cancer. We found that nanobubbles can safely and effectively label cell therapy products and such nanobubble labeled cell therapy products can be non-invasively monitored upon administration to a subject in need thereof using clinical ultrasound to determine trafficking and persistence of the cell therapy products in the subject's vasculature and tissues. This technology was found to be applicable to any adoptively infused immune cell therapy products, such as T-cells and natural killer (NK) cells.

In some embodiments, at least about 10%, at least about 20%, at least about 30%, at least about 40%, at least about 50%, at least about 60%, at least about 70%, or more immune cells can include or be intracellularly labeled with the nanobubbles.

In some embodiments, at least about 40%, at least about 50%, at least about 60%, at least about 70%, at least about 80%, or at least about 90% of the enriched population of immune cells include T-cells and/or natural killer (NK) cells that are optionally genetically modified.

In other embodiments, at least about 40%, at least about 50%, at least about 60%, at least about 70%, at least about 80%, or at least about 90% of the enriched population of immune cells are NK cells genetically modified to express one or more proteins capable of providing cytokine support. The one or more proteins capable of providing cytokine support are selected from mbIL-15, soluble IL-15, soluble IL-21, mbIL-21, mb-IL-2, or soluble IL-2.

In other embodiments, at least about 40%, at least about 50%, at least about 60%, at least about 70%, at least about 80%, or at least about 90% of the immune cells are NK cells genetically modified to express one or more proteins capable of inhibiting TGFβ signaling.

In other embodiments, at least about 40%, at least about 50%, at least about 60%, at least about 70%, at least about 80%, or at least about 90% of the enriched population of immune cells are CD4+ T-cells and/or CD8+ T-cells, which are optionally genetically modified. For example, at least about 40%, at least about 50%, at least about 60%, at least about 70%, at least about 80%, or at least about 90% of the immune cells can be chimeric antigen receptor (CAR)-CD4+ T cells and/or CAR-CD8+ T cells.

In some embodiments, the immune cells are isolated and expanded from a subject with cancer.

In some embodiments, the enriched population of immune cells includes an amount of intracellular nanobubbles effective to detect the enriched population of cells by ex vivo ultrasound contrast imaging upon administration of the enriched population of cells to a subject. For example, each of immune cells can includes at least about 25, at least about 50, at least about 100, at least about 200, at least about 500, at least about 1000, at least about 2000, at least about 5000, at least about 10,000, or more intracellular nanobubbles.

In some embodiments, each of the nanobubbles can include a lipid membrane that defines an internal void, which includes at least one gas. The gas can include, for example, perfluorocarbon gas. The lipid membrane can further include at least one of glycerol, propylene glycol, pluronic (poloxamer), alcohols or cholesterols, that change the modulus and/or interfacial tension of the nanobubble membrane.

In some embodiments, the lipid membrane can include a mixture of phospholipids having varying acyl chain lengths. For example, the lipid membrane can include a mixture of at least two of dipalmitoylphosphatidylcholine (DPPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

In some embodiments, the mixture of lipids includes at least about 50% by weight of dibehenoylglycerophosphocoline (DBPC) and less than about 50% by weight of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof.

In some embodiments, the mixture of phospholipids can include dibehenoylglycerophosphocoline (DBPC), dipalmitoylphosphatidic acid (DPPA), dipalmitoylphosphatidylethanolamine (DPPE), and distearoylphosphatidylethanolamine-N-methoxy-polyethylene glycol (DSPE-mPEG) at a ratio of about 6:1:1:1.

In other embodiments, the membrane of each nanobubble can consist essentially of dibehenoylglycerophosphocoline (DBPC), dipalmitoylphosphatidic acid (DPPA), dipalmitoylphosphatidylethanolamine (DPPE), and PEG functionalized distearoylphosphatidylethanolamine (DSPE), propylene glycol, and glycerol.

In some embodiments, the nanobubbles used to label the immune cells can have an average diameter of about 30 nm to about 600 nm, about 50 nm to about 500 nm, or about 100 nm to about 400 nm.

In some embodiments, the nanobubbles can include at least one targeting moiety that is linked to the membrane of each nanobubble. The targeting moiety can be selected from the group consisting of polypeptides, polynucleotides, small molecules, elemental compounds, antibodies, and antibody fragments. The targeting moiety can target, for example, a surface protein, molecule, or receptor of the immune cells and promote uptake of the nanobubbles by the immune cells.

Other embodiments described herein relate method that includes providing an adoptive immunotherapy composition as described herein, administering the adoptive immunotherapy composition to a subject, and generating at least one image of a region of interest (ROI) of the subject by ultrasound contrast imaging immune cells of the adoptive immunotherapy composition in the ROI of the subject.

In some embodiments, the subject has cancer and the ROI of interest includes cancerous cells or tissue in the subject.

In some embodiments, the ultrasound contrast imaging can include applying ultrasound energy to the ROI at a duty cycle of about 1% to about 100%, an ultrasound frequency of about 0.2 kHz to about 50 MHz, an intensity of about 0.1 W/cm² to about 5 W/cm², a pressure amplitude of about 50 kPa to about 10 MPa, and a time of about 1 minute to about 30 minutes.

In some embodiments, the adoptive immunotherapy composition administered to the subject can include at least about 1 million immune cells, at least about 2 million immune cells, at least about 3 million immune cells, at least about 4 million immune cells, at least about 5 million immune cells, or at least about 10 million immune cells.

Still other embodiments relate to a method of monitoring or tracking an adoptive immunotherapy composition administered to a subject. The method includes providing an adoptive immunotherapy composition as described herein, administering the adoptive immunotherapy composition to a subject, and generating at least one image of a region of interest (ROI) of the subject by ultrasound contrast imaging immune cells of the adoptive immunotherapy composition in the ROI of the subject. The at least one image of the ROI is indicative of whether the immune cells of the immunotherapy composition are in the ROI.

In some embodiments, the subject has cancer and the ROI of interest includes cancerous cells or tissue in the subject. In some embodiments, the ultrasound contrast imaging includes applying ultrasound energy to the ROI at a duty cycle of about 1% to about 100%, an ultrasound frequency of about 0.2 kHz to about 50 MHz, an intensity of about 0.1 W/cm² to about 5 W/cm², a pressure amplitude of about 50 kPa to about 10 MPa, and a time of about 1 minute to about 30 minutes.

In some embodiments, the adoptive immunotherapy composition administered to the subject can include at least about 1 million immune cells, at least about 2 million immune cells, at least about 3 million immune cells, at least about 4 million immune cells, at least about 5 million immune cells, or at least about 10 million immune cells.

Other embodiments related to a method of treating cancer in a subject in need thereof. The method includes administering a first dose of the adoptive immunotherapy composition as described herein to the subject and generating at least one image of a region of interest (ROI) of the subject by ultrasound contrast imaging immune cells of the adoptive immunotherapy composition in the ROI of the subject. The ROI includes cancerous cells or tissue in the subject and at least one image of the ROI is indicative of whether the immune cells of the immunotherapy composition are in the ROI.

In some embodiments, the ultrasound contrast imaging includes applying ultrasound energy to the ROI at a duty cycle of about 1% to about 100%, an ultrasound frequency of about 0.2 kHz to about 50 MHz, an intensity of about 0.1 W/cm² to about 5 W/cm², a pressure amplitude of about 50 kPa to about 10 MPa, and a time of about 1 minute to about 30 minutes.

In some embodiments, the adoptive immunotherapy composition administered to the subject can include at least about 1 million immune cells, at least about 2 million immune cells, at least about 3 million immune cells, at least about 4 million immune cells, at least about 5 million immune cells, or at least about 10 million immune cells.

In some embodiments, a second dose of the immunotherapy composition can be administered to the subject if the amount of immune cells in the ROI after the first dose is less than a control amount.

Still other embodiments relate to a method of generating an adoptive immunotherapy composition. The method can include mixing an enriched population of immune cells with a plurality of nanobubbles and incubating the mixture of immune cells and the plurality of nanobubbles until at least about 10%, at least about 20%, at least about 30%, at least about 40%, at least about 50%, at least about 60%, at least about 70%, or more of the immune cells internalize the nanobubbles to provide immune cells intracellularly labeled with nanobubbles. Following incubation, nanobubbles not internalized by the immune cells can be removed from the mixture.

### BRIEF DESCRIPTION OF DRAWINGS

FIG. 1 illustrate ultrasound contrast (UC) images of CAR T-cells internally labeled with 1,000, 5,000, 10,000, and 20,000 nanobubbles (NBs)/cell after 2 hours incubation at an MI of 0.19 and 0.32.

FIG. 2 illustrates a graph showing signal enhancement of CAR T-cells internally labeled with 10,000 nanobubbles (NBs)/cell after 0.5 hours, 1 hour, 2 hours, and 4 hours incubation at an MI of 0.19 and 0.32.

FIG. 3 illustrates confocal images of CAR T-cells internally labeled with 10,000 NBs/cells.

FIG. 4 illustrates plots showing cytotoxicity of CAR T-cells internally labeled with 1,000, 5,000, 10,000, and 20,000 NBs/cell 24 hours post-ultrasound.

FIG. 5 illustrates plots showing NB labelling by ultrasound does not impact CAR T-cell differentiation.

FIG. 6 illustrates images of heart/lung of mice intravenously injected with 200 L of 20M CAR T-cells internally labeled with Cy5.5-labeled NBs 2 hours, 24 hours, 48 hours, and 72 hours post injection.

FIG. 7 illustrates images of liver of mice intravenously injected with 200 μL of 20M CAR T-cells internally labeled with Cy5.5-labeled NBs 2 hours, 24 hours, 48 hours, and 72 hours post injection.

FIG. 8 illustrates images of right kidney of mice intravenously injected with 200 L of 20M CAR T-cells internally labeled with Cy5.5-labeled NBs 2 hours, 24 hours, 48 hours, and 72 hours post injection.

FIG. 9 illustrates plots of signal after background subtraction of heart/lung, liver, and kidney of mice intravenously injected with 200 μL of 20M CAR T-cells internally labeled with Cy5.5-labeled NBs 2 hours, 24 hours, 48 hours, and 72 hours post injection.

FIG. 10 illustrates UC images of liver of mice intravenously injected with 200 L of 20M CAR T-cells internally labeled with Cy5.5-labeled NBs 48 hours and 72 hours post injection.

FIG. 11 illustrates UC images of right kidney of mice intravenously injected with 200 μL of 20M CAR T-cells internally labeled with Cy5.5-labeled NBs 48 hours and 72 hours post injection.

FIG. 12 illustrates plots of signal after background subtraction of heart/lung, liver, and kidney of mice intravenously injected with 200 μL of 20M CAR T-cells internally labeled with Cy5.5-labeled NBs 48 hours and 72 hours post injection.

FIG. 13 illustrates images of tumor mice intravenously injected with 200 μL of 20M CAR T-cells CFSE labeled and internally labeled with Cy5.5-labeled NBs 48 hours and 72 hours post injection.

FIG. 14 illustrates images of heart/lung of tumor mice intravenously injected with 200 μL of 20M CAR T-cells internally labeled with Cy5.5-labeled NBs 2 hours, 24 hours, 48 hours, and 72 hours post injection.

FIG. 15 illustrates images of liver of tumor mice intravenously injected with 200 L of 20M CAR T-cells internally labeled with Cy5.5-labeled NBs 2 hours, 24 hours, 48 hours, and 72 hours post injection.

FIG. 16 illustrates images of right kidney of tumor mice intravenously injected with 200 μL of 20M CAR T-cells internally labeled with Cy5.5-labeled NBs 2 hours, 24 hours, 48 hours, and 72 hours post injection.

FIG. 17 illustrates images of peritoneum of tumor mice intravenously injected with 200 μL of 20M CAR T-cells internally labeled with Cy5.5-labeled NBs 2 hours, 24 hours, 48 hours, and 72 hours post injection.

FIG. 18 illustrates plots of signal after background subtraction of heart/lung, liver, and kidney of mice intravenously injected with 200 μL of 20M CAR T-cells internally labeled with Cy5.5-labeled NBs 2 hours, 24 hours, 48 hours, and 72 hours post injection.

FIG. 19 illustrates a graph of detected signal of heart, kidney, liver, lung, spleen, and peritoneum of mice intravenously injected with 200 μL of 20M CAR T-cells internally labeled with Cy5.5-labeled NBs 48 hours and 72 hours post injection.

FIG. 20 illustrates ultrasound contrast (UC) images of natural killer (NK) cells internally labeled with 1,000, 5,000, 10,000, and 20,000 nanobubbles (NBs)/cell after 2 hours and 24 hours incubation at an MI of 0.19, 0.32, and 0.52.

FIG. 21 illustrates graphs showing signal enhancement of 1M, 5M, and 10M NK cells internally labeled with 10,000 nanobubbles (NBs)/cell after 24 hours incubation at an MI of 0.19, 0.32, and 0.52.

FIG. 22 illustrates US images of kidney of tumor mice intravenously injected with 200 μL of 5-10M NK-cells internally labeled with Cy5.5-labeled NBs 14 minutes, 30 minutes, 3 hours, 6 hours, and 24 hours post injection.

FIG. 23 illustrates US images of liver of tumor mice intravenously injected with 200 μL of 5-10M NK-cells internally labeled with Cy5.5-labeled NBs 5 minutes, 30 minutes, 3 hours, 6 hours, and 24 hours post injection.

FIG. 24 illustrates US images of heart/lung of tumor mice intravenously injected with 200 μL of 5-10M NK-cells internally labeled with Cy5.5-labeled NBs 30 minutes, 3 hours, 6 hours, and 24 hours post injection.

FIG. 25 illustrates a graph of detected US signal of heart, lung, kidney, and liver of tumor mice intravenously injected with 200 μL of 5-10M NK-cells internally labeled with Cy5.5-labeled NBs 2 minutes, 30 minutes, 3 hours, 6 hours, and 24 hours post injection.

FIG. 26 illustrates plots of detected US signal of heart, lung, kidney, and liver of tumor mice intravenously injected with 200 μL of 5-10M NK-cells internally labeled with Cy5.5-labeled NBs up to 24 hours post injection.

FIG. 27 illustrates plots of detected US signal of heart, lung, kidney, and liver of tumor mice intravenously injected with 200 μL of 1M NK-cells internally labeled with Cy5.5-labeled NBs up to 24 hours post injection.

FIG. 28 illustrates plots of detected US signal of heart, lung, kidney, and liver of tumor mice intravenously injected with 200 μL of 5-10M NK-cells up to 24 hours post injection.

### DETAILED DESCRIPTION

Unless defined otherwise, all technical and scientific terms used herein have the same meaning as commonly understood by one of ordinary skill in the art to which this invention belongs.

As used herein, each of the following terms has the meaning associated with it in this section.

As used herein and in the appended claims, the singular forms “a”, “an”, and “the” include plural references unless the context clearly dictates otherwise. Furthermore, the terms “a” (or “an”). “one or more” and “at least one” can be used interchangeably herein. The terms “comprising”, “including”, “having” and “constructed from” can also be used interchangeably.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody” as used herein, refers to an immunoglobulin molecule, which is able to specifically bind to a specific epitope on an antigen. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1988; Houston et al., 1988; Bird et al., 1988).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of cells of the invention in prevention of the occurrence of tumor in the first place.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, melanoma, lung cancer and the like.

The term “Chimeric Antigen Receptor” or alternatively a “CAR” refers to a set of polypeptides, typically two in the simplest embodiments, which when in a T cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule. In some embodiments, the set of polypeptides are in the same polypeptide chain (e.g., comprise a chimeric fusion protein). In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one embodiment, the stimulatory molecule of the CAR is the zeta chain associated with the T cell receptor complex. In one aspect, the cytoplasmic signaling domain comprises a primary signaling domain (e.g., a primary signaling domain of CD3-zeta). In one embodiment, the cytoplasmic signaling domain further comprises one or more functional signaling domains of at least one costimulatory molecule as defined below.

In one embodiment, the CAR comprises a chimeric fusion protein comprising an extracellular antigen binding domain, a transmembrane domain and an intracellular signaling domain comprising a functional signaling domain of a stimulatory molecule. In one embodiment, the CAR comprises a chimeric fusion protein comprising an extracellular antigen binding domain, a transmembrane domain and an intracellular signaling domain comprising a functional signaling domain of a co-stimulatory molecule and a functional signaling domain of a stimulatory molecule. In one embodiment, the CAR comprises a chimeric fusion protein comprising an extracellular antigen binding domain, a transmembrane domain and an intracellular signaling domain comprising two functional signaling domains of one or more co-stimulatory molecule(s) and a functional signaling domain of a stimulatory molecule. In one embodiment, the CAR comprises a chimeric fusion protein comprising an extracellular antigen binding domain, a transmembrane domain and an intracellular signaling domain comprising at least two functional signaling domains of one or more co-stimulatory molecule(s) and a functional signaling domain of a stimulatory molecule.

The term “signaling domain” refers to the functional portion of a protein which acts by transmitting information within the cell to regulate cellular activity via defined signaling pathways by generating second messengers or functioning as effectors by responding to such messengers.

The term “intracellular signaling domain,” as the term is used herein, refers to an intracellular portion of a molecule. The intracellular signaling domain can generate a signal that promotes an immune effector function of the CAR containing cell, e.g., a CAR T cell. Examples of immune effector function, e.g., in a CAR T cell, include cytolytic activity and helper activity, including the secretion of cytokines. In embodiments, the intracellular signaling domain is the portion of a protein which transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CAR T, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3ζ, FcRγ, FcRβ, CD3γ, CD3δ, CD3ε, CD5, CD22, CD79a, CD79b, CD278 (“ICOS”), CD66d, CD32, DAP10, and DAP12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to an intracellular portion of a costimulatory molecule. The intracellular signaling domain can comprise the entire intracellular portion, or the entire native intracellular signaling domain, of the molecule from which it is derived, or a functional fragment thereof.

The intracellular signaling domain can comprise the entire intracellular portion, or the entire native intracellular signaling domain, of the molecule from which it is derived, or a functional fragment thereof.

The term “effector function” refers to a specialized function of a cell. Effector function of a T cell, for example, may be cytolytic activity or helper activity including the secretion of cytokines.

The terms “cancer associated antigen” or “tumor antigen” interchangeably refers to a molecule (typically protein, carbohydrate or lipid) that is preferentially expressed on the surface of a cancer cell, either entirely or as a fragment (e.g., MHC/peptide), in comparison to a normal cell, and which is useful for the preferential targeting of a pharmacological agent to the cancer cell. In some embodiments, a tumor antigen is a marker expressed by both normal cells and cancer cells, e.g., a lineage marker, e.g., CD19 on B cells. In certain aspects, the tumor antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like. In some embodiments, a cancer-associated antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a cancer-associated antigen is a cell surface molecule that is inappropriately synthesized in the cancer cell, for instance, a molecule that contains deletions, additions or mutations in comparison to the molecule expressed on a normal cell. In some embodiments, a cancer-associated antigen will be expressed exclusively on the cell surface of a cancer cell, entirely or as a fragment (e.g., MHC/peptide), and not synthesized or expressed on the surface of a normal cell.

An “effective amount” as used herein, means an amount which provides a therapeutic or prophylactic benefit.

The term “expression” as used herein is defined as the transcription and/or translation of a particular nucleotide sequence driven by its promoter.

The term “specifically binds,” as used herein, is meant a molecule, such as an antibody, which recognizes and binds to another molecule or feature, but does not substantially recognize or bind other molecules or features in a sample.

The term “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated. To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupT1, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naive T cells, regulator T cells, gamma delta T cells, and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

“CD4+ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8+ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

The term “NK cell” or “Natural Killer cell” refer to a subset of peripheral blood lymphocytes defined by the expression of CD56 or CD16 and the absence of the T cell receptor (CD3). As used herein, the terms “adaptive NK cell” and “memory NK cell” are interchangeable and refer to a subset of NK cells that are phenotypically CD3- and CD56+, expressing at least one of NKG2C and CD57, and optionally, CD16, but lack expression of one or more of the following: PLZF, SYK, FcR gamma, and EAT-2. In some embodiments, isolated subpopulations of CD56+NK cells comprise expression of CD16, NKG2C, CD57, NKG2D, NCR ligands, NKp30, NKp40, NKp46, activating and inhibitory KIRs, NKG2A and/or DNAM-1. CD56+ can be dim or bright expression.

The term “NKT cells” or “natural killer T cells” refers to CD1d-restricted T cells, which express a T cell receptor (TCR). Unlike conventional T cells that detect peptide antigens presented by conventional major histocompatibility (MHC) molecules, NKT cells recognize lipid antigens presented by CD1d, a non-classical MHC molecule. Two types of NKT cells are recognized. Invariant or type I NKT cells express a very limited TCR repertoire—a canonical α-chain (Vα24-Jα18 in humans) associated with a limited spectrum of β chains (Vβ11 in humans). The second population of NKT cells, called non-classical or non-invariant type II NKT cells, display a more heterogeneous TCRαβ usage. Type I NKT cells are considered suitable for immunotherapy. Adaptive or invariant (type I) NKT cells can be identified with the expression of at least one or more of the following markers, TCR Va24-Ja18, Vb11, CD1d, CD3, CD4, CD8, aGalCer, CD161 and CD56.

The term “stable cavitation” refers to gas voids of nanobubbles that have a tendency to increase in size and vibrate without imploding. The gas voids vibrate when exposed to a pressure field but do not implode. In stable cavitation, a collection of gas voids of nanobubbles tend to operate in a relatively stable manner as long as a pressure field capable of producing rectified diffusion exists.

The term “neoplastic disorder” can refer to a disease state in a subject in which there are cells and/or tissues which proliferate abnormally. Neoplastic disorders can include, but are not limited to, cancers, sarcomas, tumors, leukemias, lymphomas, and the like.

The term “neoplastic cell” can refer to a cell that shows aberrant cell growth, such as increased, uncontrolled cell growth. A neoplastic cell can be a hyperplastic cell, a cell from a cell line that shows a lack of contact inhibition when grown in vitro, a tumor cell, or a cancer cell that is capable of metastasis in vivo. Alternatively, a neoplastic cell can be termed a “cancer cell.” Non-limiting examples of cancer cells can include melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, thymoma, lymphoma cells, melanoma cells, sarcoma cells, leukemia cells, retinoblastoma cells, hepatoma cells, myeloma cells, glioma cells, mesothelioma cells, and carcinoma cells.

The term “tumor” can refer to an abnormal mass or population of cells that result from excessive cell division, whether malignant or benign, and all pre-cancerous and cancerous cells and tissues.

The terms “treating” or “treatment” of a disease (e.g., a neoplastic disorder) can refer to executing a treatment protocol to eradicate at least one neoplastic cell. Thus, “treating” or “treatment” does not require complete eradication of neoplastic cells.

Embodiments described herein relate to an adoptive immunotherapy composition that includes an enriched population of immune cells that are labeled with nanoscale ultrasound contrast agent or nanobubbles (NBs) and to the use of the adoptive immunotherapy composition in treating cancer. We found that nanobubbles can safely and effectively label cell therapy products and such nanobubble labeled cell therapy products can be non-invasively monitored upon administration to a subject in need thereof using clinical ultrasound to determine trafficking and persistence of the cell therapy products in the subject's vasculature and tissues. This technology was found to be applicable to any adoptively infused immune cell therapy products, such as T-cells and natural killer (NK) cells.

The immune cells labeled with the nanobubbles can be obtained from a subject and then manipulated ex vivo. Sources of immune cells for ex vivo manipulation can include, e.g., autologous or heterologous donor blood, cord blood, or bone marrow. For example, the source of immune cells may be from the subject to be treated with the nanobubble labeled immune cells described herein, e.g., the subject's blood, the subject's cord blood, or the subject's bone marrow. Non-limiting examples of subjects include humans, dogs, cats, mice, rats, and transgenic species thereof. In certain exemplary embodiments, the subject is a human.

In some embodiments, immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In certain embodiments, the cells are monocytes or granulocytes, e.g., myeloid cells, macrophages, neutrophils, dendritic cells, mast cells, eosinophils, and/or basophils.

In some embodiments, the immune cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as Th1 cells, Th2 cells, Th3 cells, Th17 cells, Th9 cells, Th22 cells, follicular helper T cells, alpha/beta T cells, and delta/gamma T cells. In certain embodiments, any number of T cell lines available in the art, may be used.

In certain embodiments, the isolated immune cells can be prepared, processed, cultured, and/or engineered. In certain embodiments, preparation of the engineered cells includes one or more culture and/or preparation steps. The cells for engineering as described may be isolated from a sample, such as a biological sample, e.g., one obtained from or derived from a subject. In certain embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain embodiments, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In certain embodiments, the cells are derived from cell lines, e.g., T cell lines. The cells in certain embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In certain embodiments, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In certain embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In certain embodiments, a washing step is accomplished by tangential flow filtration (TFF) according to the manufacturer's instructions. In certain embodiments, the cells are resuspended in a variety of biocompatible buffers after washing. In certain embodiments, components of a blood cell sample are removed and the cells directly resuspended in culture media. In certain embodiments, the methods include density-based cell separation methods, such as the preparation of white blood cells from peripheral blood by lysing the red blood cells and centrifugation through a Percoll or Ficoll gradient.

In certain embodiments, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In certain embodiments, the isolation methods include the separation of different cell types based on the expression or presence in the cell of one or more specific molecules, such as surface markers, e.g., surface proteins, intracellular markers, or nucleic acid. In certain embodiments, any known method for separation based on such markers may be used. In certain embodiments, the separation is affinity- or immunoaffinity-based separation. For example, the isolation in certain embodiments includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In certain embodiments, both fractions are retained for further use. In certain embodiments, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In certain embodiments, multiple rounds of separation steps are carried out, where the positively or negatively selected fraction from one step is subjected to another separation step, such as a subsequent positive or negative selection. In certain embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In certain embodiments, one or more of the T cell populations is enriched for or depleted of cells that are positive for (marker+) or express high levels (marker^{high}) of one or more particular markers, such as surface markers, or that are negative for (marker−) or express relatively low levels (marker^{low}) of one or more markers. For example, in certain embodiments, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In certain embodiments, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In certain embodiments, cells are enriched for or depleted of cells positive or expressing high surface levels of CD 122, CD95, CD25, CD27, and/or IL7-Ra (CD 127). In certain embodiments, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In certain embodiments, T cells are separated from a PBMC sample by negative selection of markers expressed on non-T cells, such as B cells, monocytes, or other white blood cells, such as CD14. In certain embodiments, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In certain embodiments, CD8+ cells are further enriched for or depleted of naive, central memory, effector memory, and/or central memory stem cells, such as by positive or negative selection based on surface antigens associated with the respective subpopulation. In certain embodiments, enrichment for central memory T (Tcm) cells is carried out to increase efficacy, such as to improve long-term survival, expansion, and/or engraftment following administration, which in some aspects is particularly robust in such sub-populations. In certain embodiments, combining Tcm-enriched CD8+ T cells and CD4+ T cells further enhances efficacy.

In certain embodiments, memory T cells are present in both CD62L+ and CD62L− subsets of CD8+ peripheral blood lymphocytes. PBMC can be enriched for or depleted of CD62L−CD8+ and/or CD62L+CD8+ fractions, such as using anti-CD8 and anti-CD62L antibodies. In certain embodiments, a CD4+ T cell population and a CD8+ T cell sub-population, e.g., a sub-population enriched for central memory (Tcm) cells. In certain embodiments, the enrichment for central memory T (Tcm) cells is based on positive or high surface expression of CD45RO, CD62L, CCR7, CD28, CD3, and/or CD 127; in some aspects, it is based on negative selection for cells expressing or highly expressing CD45RA and/or granzyme B. In certain embodiments, isolation of a CD8+ population enriched for TCM cells is carried out by depletion of cells expressing CD4, CD14, CD45RA, and positive selection or enrichment for cells expressing CD62L. In certain embodiments, enrichment for central memory T (Tcm) cells is carried out starting with a negative fraction of cells selected based on CD4 expression, which is subjected to a negative selection based on expression of CD14 and CD45RA, and a positive selection based on CD62L. Such selections in certain embodiments are carried out simultaneously and in other aspects are carried out sequentially, in either order. In certain embodiments, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+T helper cells are sorted into naive, central memory, and effector cells by identifying cell populations that have cell surface antigens. CD4+ lymphocytes can be obtained by standard methods. In certain embodiments, naive CD4+T lymphocytes are CD45RO−, CD45RA+, CD62L+, CD4+ T cells. In certain embodiments, central memory CD4+ cells are CD62L+ and CD45RO+. In certain embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CD8. In certain embodiments, the antibody or binding partner is bound to a solid support or matrix, such as a magnetic bead or paramagnetic bead, to allow for separation of cells for positive and/or negative selection.

In certain embodiments, the cells are incubated and/or cultured prior to or in connection with genetic engineering. The incubation steps can include culture, cultivation, stimulation, activation, and/or propagation. In certain embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In certain embodiments, the stimulating agents include IL-2 and/or IL-15, for example, an IL-2 concentration of at least about 10 units/mL.

In certain embodiments, T cells are isolated from peripheral blood by lysing the red blood cells and depleting the monocytes, for example, by centrifugation through a PERCOLL gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in certain embodiments, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In some embodiments, the processes described herein produce cell cultures and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% CD4/C8 T cells. In preferred embodiments, the cells of the cell cultures or cell populations comprise human cells.

In some embodiments, CD4/CD8 T cells are genetically modified with a nucleotide sequence encoding an antigen-specific receptor targeting (e.g., specifically binding to or recognizing) an antigen, such as a disease-specific target antigen corresponding to the disease or condition to be treated. In some embodiments, the CD4/CD8 T cells are modified to include one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, the genetic modification of the CD4/CD8 T cells may be performed by transduction, transfection or electroporation. Transduction can performed with lentiviruses, γ-, α-retroviruses or adenoviruses or with electroporation or transfection by nucleic acids (DNA, mRNA, miRNA, antagomirs, ODNs), proteins, site-specific nucleases (zinc finger nucleases, TALENs, CRISP/R), self-replicating RNA viruses (e.g., equine encephalopathy virus) or integration-deficient lentiviral vectors. For example, genetic modification of the CD4/CD8 T cells can be performed by transducing the CD4/CD8 T cells with lentiviral vectors

In some embodiments, the genetically engineered antigen receptor can include a T cell receptor (TCR) or components thereof, or a functional non-TCR antigen recognition receptor, such as chimeric antigen receptor (CAR), including chimeric activating receptors and chimeric costimulatory receptors. In some embodiments, the genetically engineered antigen receptor is capable of inducing an activating signal to the CD4/CD8 T cells. In some embodiments, the genetically engineered antigen receptor contains an extracellular antigen recognition domain which specifically binds to a target antigen at a dissociation constant (K_{D}) of at least 10⁻⁸M, at least 10⁻⁷M, at least 10⁻⁶M, at least 10⁻⁵M, or at least 10⁻⁴M.

In some embodiments, the genetically engineered antigen receptors include recombinant T cell receptors (TCRs) and/or TCRs cloned from naturally occurring T cells and/or pairs of chains of TCRs cloned from naturally occurring T cells. Exemplary antigen receptors, including CARs and recombinant TCRs, as well as methods for engineering and introducing the receptors into cells, include those described, for example, in international patent application publication numbers WO2000014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the genetically engineered antigen receptors include a CAR as described in U.S. Pat. No. 7,446,190, and those described in International Patent Application Publication No.: WO/2014055668 A1.

In general, TCRs contain a variable α and β chain (also known as TCRα and TCRβ, respectively) or a variable γ and δ chain (also known as TCRγ and TCRδ, respectively) or antigen-binding portion(s) thereof, and in general are capable of specifically binding to an antigen peptide bound to a MHC receptor. Thus, TCR T cells can provide specificity and reactivity toward a selected target, but in an MHC-restricted manner.

In some embodiments, the TCR is in the up form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). For example, in some aspects, each chain of the TCR can possess one N-terminal immunoglobulin variable domain, one immuno-globulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e., MHC-peptide complex. An “antigen-binding portion” or antigen-binding fragment” of a TCR, which can be used interchangeably, refers to a molecule that contains a portion of the structural domains of a TCR, but that binds the antigen (e.g., MHC-peptide complex) to which the full TCR binds. In some cases, an antigen-binding portion contains the variable domains of a TCR, such as variable α chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jares et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Va or V13; typically amino acids 1 to 116 based on Kabat numbering Kabat et al., “Sequences of Proteins of Immunological Interest, U.S. Dept. Health and Human Services, Public Health Service National Institutes of Health, 1991, 5th ed.) at the N-terminus, and one constant domain (e.g., α-chain constant domain or C_{α} typically amino acids 117 to 259 based on Kabat, β-chain constant domain or C_{β}, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, a TCR for a target antigen (e.g., a cancer antigen) is identified and introduced into the cells. In some embodiments, a TCR for a target antigen also specifically binds to, e.g., is cross-reactive with, one or more peptide epitopes of one or more other antigens, such as those that are related to (e.g., by way of sharing sequence or structural similarity with) the target antigen. The crossreactive antigen may have an epitope that is the same as or has one or more amino acid differences as compared to the target antigen, such as one, two, or three differences. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells, such as from a T cell (e.g., cytotoxic T cell), T-cell hybridomas or other publicly available source. In some embodiments, the T-cells can be obtained from in vivo isolated cells. In some embodiments, a T cell clone, such as a high-affinity T cell clone can be isolated from a patient, and the TCR isolated. In some embodiments, the T-cells can be a cultured T-cell hybridoma or clone. In some embodiments, the TCR clone for a target antigen has been generated in transgenic mice engineered with human immune system genes (e.g., the human leukocyte antigen system, or HLA). See, e.g., Parkhurst et al. (2009) Clin Cancer Res. 15: 169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354. In some embodiments, the TCR or antigen-binding portion thereof can be synthetically generated from knowledge of the sequence of the TCR.

In some embodiments, after the T-cell clone is obtained, the TCR α and β chains are isolated and cloned into a gene expression vector. In some embodiments, the TCR α and β genes are linked via a picomavirus 2A ribosomal skip peptide so that both chains are coexpression. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13: 1050-1063; Frecha et al. (2010) Molecular Therapy: The Journal of the American Society of Gene Therapy. 18:1748-1757; and Hackett et al. (2010) Molecular Therapy: The Journal of the American Society of Gene Therapy. 18:674-683.

In some embodiments, the CD4/CD8 T cells method can be genetically modified with a nucleotide sequence encoding a chimeric antigen receptor (CAR). The CAR may have antigenic specificity for a cancer antigen or an infectious disease antigen.

The CARs disclosed herein comprise at least one extracellular domain capable of binding to an antigen, at least one transmembrane domain, and at least one intracellular domain.

A chimeric antigen receptor (CAR) is an artificially constructed hybrid protein or polypeptide containing the antigen binding domains of an antibody (e.g., single chain variable fragment (scFv)) linked to T-cell signaling domains via a transmembrane domain. Characteristics of CARs include their ability to redirect T-cell specificity and reactivity toward a selected target in a non-MHC-restricted manner, and exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

In some embodiments, the intracellular T cell signaling domains of the CARs can include, for example, a T cell receptor signaling domain, a T cell costimulatory signaling domain, or both. The T cell receptor signaling domain refers to a portion of the CAR comprising the intracellular domain of a T cell receptor, such as, for example, and not by way of limitation, the intracellular portion of the CD3 zeta protein. The costimulatory signaling domain refers to a portion of the CAR comprising the intracellular domain of a costimulatory molecule, which is a cell surface molecule other than an antigen receptor or their ligands that are required for an efficient response of lymphocytes to antigen.

In some embodiments, the antigen-specific receptor used in the CD4/CD8 T-cell population(s) as disclosed herein, includes a target-specific binding element otherwise referred to as an antigen binding domain or moiety. The choice of domain depends upon the type and number of ligands that define the surface of a target cell. For example, the antigen binding domain may be chosen to recognize a ligand that acts as a cell surface marker on target cells associated with a particular disease state. In some embodiments, a target antigen that is expressed on or in, specifically expressed on or in, or associated with, the particular disease state or condition may be referred to as a “disease-specific target” “disease-specific antigen” or “disease-specific antigen”. Thus, examples of cell surface markers that may act as ligands for the antigen binding domain in the genetically engineered antigen-specific receptor include those associated with viral, bacterial and parasitic infections, autoimmune disease and cancer cells.

In one embodiment, the antigen-specific receptor can be engineered to target a tumor antigen of interest by way of engineering a desired antigen binding domain that specifically binds to an antigen on a tumor cell. Tumor antigens are proteins that are produced by tumor cells that elicit an immune response, particularly T-cell mediated immune responses. The selection of the antigen binding domain will depend on the particular type of cancer to be treated. Tumor antigens are well known in the art and include, for example, a glioma-associated antigen, carcinoembryonic antigen (CEA), CEACAM5, beta-human chorionic gonadotropin, afetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, GD-2, prominin-1 (CD133), folate receptor alpha (FRa), and mesothelin. The tumor antigens disclosed herein are merely included by way of example. The list is not intended to be exclusive and further examples will be readily apparent to those of skill in the art.

In one embodiment, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20, CD22, and CD37 are other candidates for target antigens in B-cell lymphoma. Some of these antigens (CEA, HER-2, CD19, CD20, CD22, idiotype) have been used as targets for passive immunotherapy with monoclonal antibodies with limited success.

The type of tumor antigen may also be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSAs or TAAs include the following: Differentiation antigens such as MART-1/MelanA (MART-I), gp100 (Pmel 17), tyrosinase, TRP-1, TRP-2 and tumor-specific multi-lineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In a preferred embodiment, the antigen binding domain portion of the antigen-specific receptor targets an antigen that includes but is not limited to CD19, CD20, CD22, ROR1, Mesothelin, CD33, c-Met, PSMA, Glycolipid F77, EGFRvIII, GD-2, MY-ESO-1 TCR, MAGE A3 TCR, and the like.

Depending on the desired antigen to be targeted, the antigen-specific receptor can be engineered to include the appropriate antigen bind domain that is specific to the desired antigen target. For example, if CD19 is the desired antigen that is to be targeted, an antibody for CD19 can be used as the antigen bind domain incorporation into the CAR.

In one exemplary embodiment, the antigen binding domain portion of the antigen-specific receptor is an antigen-specific receptor, such as a CAR, that targets CD19. Preferably, the antigen binding domain in the CAR is anti-CD19 scFV.

In another embodiment, scFvs can be replaced with a nanobody, such as a nanobody derived from camelids.

In other embodiments, an antigen-specific receptor can be expressed that is capable of binding to a non-TSA or non-TAA including, for example and not by way of limitation, an antigen derived from Retroviridae (e.g., human immunodeficiency viruses such as HIV-1 and HIV-LP), Picornaviridae (e.g., poliovirus, hepatitis A virus, enterovirus, human coxsackievirus, rhinovirus, and echovirus), rubella virus, coronavirus, vesicular stomatitis virus, rabies virus, ebola virus, parainfluenza virus, mumps virus, measles virus, respiratory syncytial virus, influenza virus, hepatitis B virus, parvovirus, Adenoviridae, Herpesviridae [e.g., type 1 and type 2 herpes simplex virus (HSV), varicella-zoster virus, cytomegalovirus (CMV), and herpes virus], Poxviridae (e.g., smallpox virus, vaccinia virus, and pox virus), or hepatitis C virus, or any combination thereof.

In other embodiments, an antigen-specific receptor can be expressed that is capable of binding to an antigen derived from a bacterial strain of Staphylococci, Streptococcus, Escherichia coli, Pseudomonas, or Salmonella. Particularly, there is provided an antigen-specific receptor capable of binding to an antigen derived from an infectious bacterium, for example, Helicobacter pyloris, Legionella pneumophilia, a bacterial strain of Mycobacteria sps. (e.g., M. tuberculosis, M. avium, M. intracellulare, M. kansaii, or M. gordonea), Staphylococcus aureus, Neisseria gonorrhoeae, Neisseria meningitides, Listeria monocytogenes, Streptococcus pyogenes, Group A Streptococcus, Group B Streptococcus (Streptococcus agalactiae), Streptococcus pneumoniae, or Clostridium tetani, or a combination thereof.

The one or more transmembrane domains fused to the extracellular domain of an antigen-specific receptor, such as CAR, can be derived either from a natural or from a synthetic source. Where the source is natural, the domain may be derived from any membrane-bound or transmembrane protein. Transmembrane regions of particular can be derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In one embodiment, the transmembrane domain in the antigen-specific receptor, such as CAR, can be a CD8 transmembrane domain. Other non-limiting examples of transmembrane domains for use in the CARs disclosed herein include the TNFRSF16 and TNFRSF19 transmembrane domains may be used to derive the TNFRSF transmembrane domains and/or linker or spacer domains disclosed including, in particular, those other TNFRSF members listed within the tumor necrosis factor receptor superfamily.

In some embodiments, the CARs expressed in the CD4/CD8 T-cell population(s) as disclosed herein, include a spacer domain that can be arranged between the extracellular domain and the TNFRSF transmembrane domain, or between the intracellular domain and the TNFRSF transmembrane domain. The spacer domain means any oligopeptide or polypeptide that serves to link the TNFRSF transmembrane domain with the extracellular domain and/or the TNFRSF transmembrane domain with the intracellular domain. The spacer domain can include up to 300 amino acids, 10 to 100 amino acids, or 25 to 50 amino acids.

In several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,5667, 498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference herein in its entirety.

The spacer domain preferably has a sequence that promotes binding of an antigen-specific receptor, such as CAR, with an antigen and enhances signaling into a cell. Examples of an amino acid that is expected to promote the binding include cysteine, a charged amino acid, and serine and threonine in a potential glycosylation site, and these amino acids can be used as an amino acid constituting the spacer domain.

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone can be insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the CARS disclosed herein include those derived from TCRζ (CD3ζ), FcRα, FcRβ, CD3γ, CD3δ, CD3ε, CD5, CD22, CD79a, CD79b, and CD66d. In one embodiment, the cytoplasmic signaling molecule in the CAR comprises a cytoplasmic signaling sequence derived from CD3 zeta. The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the intracellular domain is designed to comprise the signaling domain of CD3-ζ and the signaling domain of CD28. In another embodiment, the intracellular domain is designed to comprise the signaling domain of CD3-zeta and the signaling domain of 4-1BB. In yet another embodiment, the intracellular domain is designed to comprise the signaling domain of CD3-ζ and the signaling domain of CD28 and 4-1BB.

Exemplary CARs include those described in International Patent Application Publication No. WO 2011041093 and International Application No. PCT/US 12/29861, each of which is incorporated herein by reference. Exemplary TCRs include those described in U.S. Pat. Nos. 7,820,174; 8,088,379; 8,216,565; U.S. Patent Application Publication No. 20090304657; and International Patent Application Publication Nos. WO 2012040012 and WO 2012054825, each of which is incorporated herein by reference. The cells may be transduced using any suitable method known in the art, for example, as described in Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994.

In some embodiments, improved selectivity and specificity is achieved through strategies targeting multiple antigens. Such strategies generally involve multiple antigen binding domains, which typically are present on distinct genetically engineered antigen receptors and specifically bind to distinct antigens. Thus, in some embodiments, the CD4/CD8 T cells are engineered with the ability to bind more than one antigen. In some aspects, a plurality of genetically engineered antigen receptors are introduced into the cell, which specifically bind to different antigens, each expressed in or on the disease or condition to be targeted with the cells or tissues or cells thereof. Such features can in some aspects address or reduce the likelihood of off-target effects. For example, where a single antigen expressed in a disease or condition is also expressed on or in non-diseased or normal cells, such multi-targeting approaches can provide selectivity for desired cell types by requiring binding via multiple antigen receptors in order to activate the cell or induce a particular effector function.

In some embodiments, at least about 40%, at least about 50%, at least about 60%, at least about 70%, at least about 80%, or at least about 90% of the enriched population of immune cells are CD4+ T-cells and/or CD8+ T-cells, which are optionally genetically modified. For example, at least about 40%, at least about 50%, at least about 60%, at least about 70%, at least about 80%, or at least about 90% of the immune cells can be chimeric antigen receptor (CAR)-CD4+ T cells and/or CAR-CD8+ T cells.

In some embodiments, the CD4/CD8 T cells include other genetically engineered antigen-specific receptor, such as a costimulatory receptor, that specifically binds to another antigen and is capable of inducing a costimulatory signal to the cell. In some aspects, such another target antigen and the first target antigen recognized by the first antigen-specific receptor are distinct.

In some embodiments, the other genetically engineered antigen-specific receptor is one that is not expressed or is not specifically expressed or associated with the disease or condition. In some aspects the other genetically engineered antigen-specific receptor is one that may be expressed or associated with another cancer or infectious disease that is not targeted by the first target antigen, and in some aspects another antigen is not expressed or specifically expressed or associated with any cancer or infectious disease.

In some embodiments, ligation of the first genetically engineered antigen-specific receptor and the other engineered antigen-specific receptor (e.g., a second engineered antigen-specific receptor) induces a response in the CD4/CD8 T cell, which response is not induced by ligation of either of the genetically engineered antigen receptors alone. In some embodiments, the response is selected from the group consisting of proliferation, secretion or a cytokine, and cytotoxic activity.

In certain embodiments, CD4/CD8 T cells are further modified in order to increase their therapeutic or prophylactic efficacy. For example, in some embodiments, the engineered antigen-specific receptor expressed by the CD4/CD8 T cells can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 1 1 1 (1995), and U.S. Pat. No. 5,087,616.

In some embodiments, CD4/CD8 T cells are further modified in order to enhance T cell trafficking/homing to targeted sites, such as tumor sites. For example, genetically engineered T cells expressing an antigen-specific receptor can be further modified with chemokine receptors that specifically bind chemokines produced by tumors. In some embodiments, genetically engineered T cells expressing an antigen-specific receptor can be further modified to coexpress CCR2 and/or CCR4. Genetically engineered T cells expressing VEGFR-1 have been shown to delay tumor growth and formation and suppress metastasis in tumor models. Therefore, in some embodiments, CD4/CD8 T cells are further modified to coexpress VEGFR-1.

Various immunosuppressive cytokines such as transforming growth factor (TGF)-β and IL-10, are involved in the inhibition of engineered T cell based cancer immunotherapy. In some embodiments, genetically engineered T cells expressing an antigen-specific receptor can express a dominant-negative TGF-β and/or IL-10 receptor. IL-2, IL-4, IL-7, IL-15, and IL-21 have been shown to mitigate the effects of immunosuppressive factors in the tumor microenvironment and enhance genetically engineered T cell efficacy. Therefore, CD4/CD8 T cells can be further genetically modified to express one or more of IL-2, IL-4, IL-7, IL-15, and IL-21.

Programmed cell death protein-1 (PD-1) has been implemented as a target to promote genetically engineered T cell efficacy. In some embodiments, CD4/CD8 T cells are further modified to genetically deplete PD-1. In some embodiments, CD4/CD8 T cells are further modified to coexpress PD-1 antibody.

In other embodiments, the immune cells include natural killer (NK) cells. Human NK cells are typically characterized as lymphocytes expressing CD56 or CD16 and lacking CD3 expression, and are estimated to comprise up to about one-third of peripheral blood lymphocytes in normal subjects. Unlike T-cells, NK cells recognize targets in a major histocompatibility complex (MHC)-unrestricted manner.

There are various known methods for isolating NK cells from peripheral blood. Generally, to isolate natural killer cells from peripheral blood, PBMCs are separated into lymphocytes and monocytes, and the lymphocytes are further divided into T cells, B cells, and natural killer cells for isolation.

The peripheral blood mononuclear cells can be obtained from human blood collected using known methods such as the Ficoll-Hypaque density gradient method. PMBCs may be obtained from a healthy person, a patient at risk of cancer, or a cancer patient. The PBMCs used herein can be, but do not necessarily need to be, autologous; allogeneic PBMCs may also be used to induce and proliferate the NK cells for anti-cancer immunotherapy according to the present disclosure.

In some embodiments, NK cells may be derived from a subject and grown in vitro to provide a population of NK cells for use in the present disclosure. In deriving NK cells from a subject, the cells may come from stem cells or they may be collected from a living donor. In a preferred aspect, the NK cells employed herein are collected from a living donor. In certain aspects, the living donor many be a human living donor. In an alternative embodiment, a NK cell known in the art that has previously been isolated and cultured may be used in the present invention. Thus an established NK cell line may be used. Many such NK cells lines are commercially available and known to those in the art.

Once isolated, NK cells can be expanded if larger numbers are desired. As used herein, “expanded” refers to the increase in number of NK cells by any method. Though several expansion platforms have been developed for NK cells, few have the potential to efficiently produce a large magnitude of highly active NK cells.

In some embodiments, the administration of the NK cell may be non-immunogenic, for example, by providing a conditioning regimen (e.g., cyclophosphamide and fludarabine) to the patient at the time of administration. The term “non-immunogenic” is thus used broadly herein to mean that when the cell is injected into or otherwise administered to a subject, it avoids detection by the body's immunological system and is not rejected or recognized as foreign. More particularly, the cell does not raise (or is not capable of raising) an immune response sufficient to lead to rejection of the cell and/or to affect the function of the cells. Thus, the cells retain cytotoxic activity in the subject, more particularly, significant or substantial or measurable cytotoxic activity against a target cell. As with any biological system, the absence of an immune response may not be absolute (or 100%), A small (or mild or minor) immune response to the NK cell (e.g., a de minimis immune response) may be tolerated, as long as the function or utility of the cells is not substantially affected (i.e., as long as the cells can still perform their function). That is, the NK cells employed herein may be “universal” in nature such that one set of donor cells can be used for virtually any patient without generating a negative immune response.

The NK cells may be autologous or allogeneic NK cell. If the NK cells are derived from an identical twin, they may be termed “syngeneic”. In particularly preferred embodiments, the NK cells employed according to the disclosed methods, including the methods of treating cancer, are allogeneic NK cells.

In some embodiments, the isolated NK cells can be expanded using feeder cells. As used herein, the term “feeder cells” refers to cells that, due to their metabolic activity, produce various metabolites to thereby assist in the proliferation of target cells, even though these cells cannot themselves proliferate.

Feeder cells as used according to the present disclosure may be any population of leukemia cells engineered to express a membrane-bound interleukin (mbIL). As used herein, the term “interleukin (IL) protein” refers to a collection of biologically active cytokines produced by immune cells such as lymphocytes, monocytes or macrophages. According to the present disclosure, the term “cytokine” refers to an immune activating cytokine (secreted protein and/or signaling molecule) that can be used to induce NK cells isolated from PBMCs. Examples of IL proteins which may be used in the present disclosure include IL-2, IL-7, IL-12, IL-15, IL-18, IL-21, Flt3-L, SCF, IL-7 and the like.

In certain aspects of the disclosure, the feeder cells are HL-60 cells or OCI-AML3 cells. In a preferred aspect, the feeder cells are OCI-AML3 cells. Preferably, the mbIL comprises one or more of IL-15 or IL-21. In a preferred aspect, the mbIL consists of, or consists essentially of, mbIL-15. In another preferred aspect, the mbIL consists of, or consists essentially of, mbIL-21.

The cells that are used as feeder cells according to the present disclosure may be non-inactivated or inactivated cells whose proliferation was inhibited prior to use. More specifically, the feeder cells may be inactivated to ensure their safety and to eliminate their potential to proliferate when employed as part of the feeding platform described herein. A common method for inactivating feeder cells comprises the step of irradiating the killer cells with gamma-rays. If non-inactivated feeder cells are used, they can be killed by natural killer cells during culture because they are tumor cells. In a preferred aspect of the present disclosure, the feeder cells are inactivated using gamma radiation prior to adding them to the cell culture comprising NK cells. In some embodiments, the feeder cells can be inactivated using 10 Gy or greater or of radiation, such as 10 Gy, 20 Gy, 30 Gy, 40 Gy, 50 Gy, 60 Gy, 70 Gy, 80 Gy, 90 Gy, 100 Gy, 110 Gy, 120, Gy, 130 Gy, 140 Gy, 150 Gy, 175 Gy, 200 Gy, 225 Gy, 250 Gy, 300 Gy, 350 Gy, 400 Gy, 450, Gy or 500 Gy. In preferred methods, the feeder cells are inactivated using 90 Gy of gamma radiation.

In some embodiments, ex vivo expansion of the NK cells employs a feeder cell line constructed from an AML cells line transduced with a membrane-bound IL protein. In some aspects of the present disclosure, OCI-AML3 cells, HL-60 cells, or combinations thereof, are employed as the feeder cell line. Preferably, OCI-AML3 cells are employed as the feeder cell line.

The presence of membrane-bound IL proteins such as membrane-bound IL-15 (mbIL-15) or membrane-bound IL-21 (mbIL-21) is believed to prevent NK cells from undergoing senescence, markedly improving their ability to expand ex vivo. There are a myriad of studies proposing how and why cytokines exert their effect on NK cell function and prevention of senescence, but a single, clear explanation has not yet emerged. The feeder cells of the present disclosure preferably comprise mbIL-15, mb-IL-21 or combinations thereof as the membrane-bound IL protein.

In some embodiments, the feeder cell line has been engineered to express mbIL-15 and/or mbIL-21. The feeder cells according can be further modified to express one or more associated accessory signaling polypeptides, cytokines or fragments thereof. Such expression may correlate with increased expression of the mbIL proteins in certain aspects.

In addition to the NKF cells, NK cells can be expanded in vivo or ex vivo in the presence of cytokine support. Cytokine support can be used to enable the cells to survive and proliferate after infusion into the patient. Exemplary cytokines for use according to the disclosed methods include IL-2, IL-15, ALT-803, hetIL-15, IL-12, IL-18, IL-21 or fragments or derivatives thereof. The present methods may comprise the use of more than one cytokine support. In preferred aspects of the present methods, NK cells may be expanded in the presence of IL-2, IL-15 or ALT-803 or other IL-15 derivatives. Alternatively, cytokine support can be provided by engineering the NK cells to express additional cytokines. For example, this can be accomplished by transducing genes for one or more of mbIL-15, soluble IL-15, soluble IL-21, mbIL-21, mbIL-2, or soluble IL-2 into the NK cells prior to or after NK cell expansion. In some aspects of the disclosure, cytokine support is provided by pharmacologic inhibitors. In some aspects of the disclosure, cytokine support is provided by genetic modification. In other aspects, cytokine support can be provided both pharmacologically and genetically.

A novel IL-15 superantagonist called ALT-803 has recently been developed that offers the potential to markedly improve the survival/proliferation of adoptively infused NK cells. (Zhu, X. et al. Novel Human Interleukin-15 Agonists. J. Immunol. 2009; 183(6):3598-3607, herein incorporated by reference). ALT-803 is a fusion protein containing a mutated IL-15 molecule fused to a portion of the IL-15 receptor (αSu/Fc fusion protein). ALT-803 exhibits greater than 25-fold enhancement in biological activity compared to IL-15 and a markedly improved half-life (25 hrs).

In certain aspects of the disclosed methods, the expanding of NK cells in the presence of NKF cells can last up from one to eight weeks. That is, the step of expanding NK cells can take, e.g., one week, two weeks, three weeks, four weeks, five weeks, six weeks, seven weeks, or eight weeks.

When NK cells are expanded for more than one week, the NKF cells may need refreshed or replenished throughout the step of expanding the NK cells. Refreshing of NKF cells can be done on an as-needed basis, preferably weekly. The amount of NKF cells for refreshment can employ the same ratio of NKF:NK cells as the starting ratio or the NKF cells can be replenished in a different ratio as needed. In some aspects of the present method, the ratio of NKF cells to NK cells of refreshment is preferably greater than or equal to about 1:1, such as about 1:1, about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1, based on the number of NK cells counted on the day of the NKF cell addition. In some aspects, a 5:1 ratio of NKF:NK cells is particularly preferred. In other aspects of the present method, the NKF:NK cell ratio is about 10:1, or greater, such as 10:1, 15:1, 20:1, 25:1 or 30:1, based on the NK cell count on the day of the NKF cell addition. In a particularly preferred embodiment, the NKF:NK cell ratio is 10:1.

In other embodiments, at least about 40%, at least about 50%, at least about 60%, at least about 70%, at least about 80%, or at least about 90% of the enriched population of immune cells are NK cells genetically modified to express one or more proteins capable of providing cytokine support. The one or more proteins capable of providing cytokine support are selected from mbIL-15, soluble IL-15, soluble IL-21, mbIL-21, mb-IL-2, or soluble IL-2. In other embodiments, at least about 40%, at least about 50%, at least about 60%, at least about 70%, at least about 80%, or at least about 90% of the immune cells are NK cells genetically modified to express one or more proteins capable of inhibiting TGFβ signaling.

In some embodiments, the immune cells can be frozen, e.g., cryopreserved, either before or after isolation, incubation, and/or engineering. In some embodiments, freezing and subsequent thawing of the immune cells can removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1: 1 with media so that the final concentration of DMSO and HSA are 10% and 4%, respectively. The cells are then frozen to −80° C. at a rate of 1° per minute and stored in the vapor phase of a liquid nitrogen storage tank.

The nanobubbles used to label the immune cells can have a membrane, such as a lipid membrane, that defines at least one internal void, which includes at least one gas. The lipid membrane can exhibit selective activation and/or cavitation to known ultrasound pressures. In some embodiments, the lipid membrane can be specifically modified to elicit cavitation at predictable pressures. This can avoid collateral damage and activation of other nanoscale gas nucleation sites. The composition of the lipid membrane used to form the nanobubbles also enables the cavitation threshold to be significantly lowered.

In some embodiments, the lipid membrane can include, for example, a plurality of lipids, an edge-activator, which is incorporated between lipids of the membrane and enhances the flexibility of the nanobubbles, a membrane stiffener, which is incorporated on an outer surface of the membrane and enhances the membranes resistance to tearing, and, other additives, such as pluronic (poloxamer), alcohols and cholesterols, that change the modulus and/or interfacial tension of the bubble shell.

In other embodiments, each of the nanobubbles can include a hydrophilic outer domain at least partially defined by hydrophilic heads of the lipid and a hydrophobic inner domain at least partially defined by hydrophobic tails of the lipid. An edge activator, such as propylene glycol, can at least partially extend between the lipids from the outer domain to the inner domain. The glycerol can be provided on the outer domain of the nanobubbles and extend partially between hydrophilic heads of the lipids. The gas, which is encapsulated by the membrane, can have a low solubility in water (e.g., hydrophobic gas) and include, for example, a perfluorocarbon, such as perfluoropropane or perfluorobutane, sulfur hexafluoride, carbon dioxide, nitrogen (N₂), oxygen (O₂), and air.

The nanobubbles can have a lipid concentration that enhances the intracellular stability of the nanobubbles. For example, a higher lipid concentration can be associated with an increase in stability the nanobubbles upon intracellular uptake by the immune cells. In some embodiments, the nanobubbles can have a lipid concentration of at least about 2 mg/ml, at least about 3 mg/ml, at least about 4 mg/ml, at least about 5 mg/ml, about 6 mg/ml, at least about 7 mg/ml, at least about 8 mg/ml, at least about 9 mg/ml, at least about 10 mg/ml, at least about 11 mg/ml, at least about 12 mg/ml or more. In other embodiments, the lipid concentration of the nanobubbles can be about 5 mg/ml to about 12 mg/ml, about 6 mg/ml to about 12 mg/ml, about 7 mg/ml to about 12 mg/ml, about 8 mg/ml to about 12 mg/ml, about 9 mg/ml to about 12 mg/ml, about 10 mg/ml to about 12 mg/ml, or at least about 10 mg/ml.

The plurality of lipids comprising the membrane or shell can include any naturally-occurring, synthetic or semi-synthetic (i.e., modified natural) moiety that is generally amphipathic or amphiphilic (i.e., including a hydrophilic component and a hydrophobic component). Examples of lipids, any one or combination of which may be used to form the membrane, can include: phosphocholines, such as 1-alkyl-2-acetoyl-sn-glycero 3-phosphocholines, and 1-alkyl-2-hydroxy-sn-glycero 3-phosphocholines; phosphatidylcholine with both saturated and unsaturated lipids, including dioleoylphosphatidylcholine, dimyristoylphosphatidylcholine, dipentadecanoylphosphatidylcholine, dilauroylphosphatidylcholine, dipalmitoylphosphatidylcholine (DPPC), dibehenoylglycerophosphocoline (DBPC), distearoylphosphatidylcholine (DSPC), and diarachidonylphosphatidylcholine (DAPC); phosphatidylethanolamines, such as dioleoylphosphatidylethanolamine, dipalmitoylphosphatidylethanolamine (DPPE), and distearoylphosphatidylethanolamine (DSPE); phosphatidylserine; phosphatidylglycerols, including distearoylphosphatidylglycerol (DSPG); phosphatidylinositol; sphingolipids, such as sphingomyelin; glycolipids, such as ganglioside GM1 and GM2; glucolipids; sulfatides; glycosphingolipids; phosphatidic acids, such as dipalmitoylphosphatidic acid (DPPA) and distearoylphosphatidic acid (DSPA); palmitic acid; stearic acid; arachidonic acid; oleic acid; lipids bearing polymers, such as chitin, hyaluronic acid, polyvinylpyrrolidone or polyethylene glycol (PEG); lipids bearing sulfonated mono-, di-, oligo- or polysaccharides; cholesterol, cholesterol sulfate, and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylaamine; cardiolipin; phospholipids with short chain fatty acids of about 6 to about 8 carbons in length; phospholipids with medium chain fatty acids of about 10 to about 16 carbons in length; phospholipids with long chain fatty acids of about 18 to about 24 carbons in length; synthetic phospholipids with asymmetric acyl chains, such as, for example, one acyl chain of about 6 carbons and another acyl chain of about 12 carbons; ceramides; non-ionic liposomes including niosomes, such as polyoxyalkylene (e.g., polyoxyethylene) fatty acid esters, polyoxyalkylene (e.g., polyoxyethylene) fatty alcohols, polyoxyalkylene (e.g., polyoxyethylene) fatty alcohol ethers, polyoxyalkylene (e.g., polyoxyethylene) sorbitan fatty acid esters (such as, for example, the class of compounds referred to as TWEEN (commercially available from ICI Americas, Inc., Wilmington, DE), glycerol polyethylene glycol oxystearate, glycerol polyethylene glycol ricinoleate, alkyloxylated (e.g., ethoxylated) soybean sterols, alkyloxylated (e.g., ethoxylated) castor oil, polyoxyethylene-polyoxypropylene polymers, and polyoxyalkylene (e.g., polyoxyethylene) fatty acid stearates; sterol aliphatic acid esters including cholesterol sulfate, cholesterol butyrate, cholesterol isobutyrate, cholesterol palmitate, cholesterol stearate, lanosterol acetate, ergosterol palmitate, and phytosterol n-butyrate; sterol esters of sugar acids including cholesterol glucuronide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, and n-octadecyl alcohol; 6-(5-cholesten-30-yloxy)-1-thio-β-D-galactopyranoside; digalactosyldiglyceride; 6-(5-cholesten-30-yloxy)hexyl-6-amino-6-deoxy-1-thio-β-D-galactopyranoside; 6-(5-cholesten-30-yloxy)hexyl-6-amino-6-deoxyl-1-thio-α-D-mannopyranoside; 12-(((7′-diethylaminocoumarin-3-yl)carbonyl)methylamino)octadecanoic acid; N-[12-(((7′-diethylaminocoumarin-3-yl)carbonyl)methylamino)octadecanoyl]-2-aminopalmitic acid; cholesteryl(4′-trimethylammonio)butanoate; N-succinyldioleoylphosphatidylethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine and palmitoylhomocysteine; and/or any combinations thereof.

In some embodiments, the plurality of lipids used to form the membrane can include a mixture of phospholipids having varying acyl chain lengths. For example, the lipids can include a mixture of at least two of dipalmitoylphosphatidylcholine (DPPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

In other embodiments, the mixture of phospholipids having varying acyl chain length can include dibehenoylglycerophosphocoline (DBPC) and one or more additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof.

In some embodiments, the mixture of phospholipids can include at least about 40%, at least about 50%, at least about 60%, at least about 70%, or at about 80%, by weight of dibehenoylglycerophosphocoline (DBPC); and less than about 60%, less than about 50%, less than about 40%, less than about 30%, or less than about 20%, by weight, of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof. The PEG can have a molecular weight of about 1000 to about 5000 Da, for example, about 2000 Da.

In some embodiments, the mixture of phospholipids can include about 40% to about 80%, about 50% to about 70%, or about 55% to about 65% (e.g., about 60%) by weight dibehenoylglycerophosphocoline (DBPC); and about 20% to about 60%, about 30% to about 50%, or about 35% to about 45% (e.g., about 40%) by weight of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof.

In other embodiments, the one or more additional phospholipids can include, consist essentially of, or consists of a combination of dipalmitoylphosphatidic acid (DPPA), dipalmitoylphosphatidylethanolamine (DPPE), and PEG functionalized distearoylphosphatidylethanolamine (DSPE)

In still other embodiments, the mixture of phospholipids can include dibehenoylglycerophosphocoline (DBPC), dipalmitoylphosphatidic acid (DPPA), dipalmitoylphosphatidylethanolamine (DPPE), and PEG functionalized distearoylphosphatidylethanolamine (DSPE) at a ratio of, for example, about 6:1:1:1 by weight.

In some embodiments, the edge-activator, which is incorporated between lipids of the membrane of each nanobubble and enhances the flexibility of the nanobubbles can include a co-surfactant, such as propylene glycol, which enhances the effectiveness of phospholipid surfactants. The edge activator can be provided in each of the nanobubbles at an amount effective to cause separation of lipid domains of the nanobubble and form defects that absorb excessive pressure, which could have caused lipid “domain” tearing. Other edge activators, which can be substituted for propylene glycol or used in combination with propylene glycol, can include cholesterol, sodium cholate, limonene, oleic acid, and/or span 80.

In some embodiments, the amount of propylene glycol provided in the nanobubbles can be about 0.05 ml to about 0.5 ml, about 0.06 ml to about 0.4 ml, about 0.07 ml to about 0.3 ml, about 0.08 ml to about 0.2 ml, or about 0.1 ml, per 1 ml of hydrated lipids.

In other embodiments, a membrane stiffener, which is incorporated on the outer surface of the membrane of each nanobubble and enhances the membranes resistance to tearing, includes glycerol. Glycerol can be provided on the membrane of each of the nanobubbles at an amount effective to stiffen the membrane and improve the membrane's resistance to lipid “domain” tearing. The amount of glycerol provided on the membranes of the nanobubbles can be about 0.05 ml to about 0.5 ml, about 0.06 ml to about 0.4 ml, about 0.07 ml to about 0.3 ml, about 0.08 ml to about 0.2 ml, or about 0.1 ml, per 1 ml of hydrated lipids.

The membranes defining the nanobubbles can be concentric or otherwise and have a unilamellar configuration (i.e., comprised of one monolayer or bilayer), an oligolamellar configuration (i.e., comprised of about two or about three monolayers or bilayers), or a multilamellar configuration (i.e., comprised of more than about three monolayers or bilayers). The membrane can be substantially solid (uniform), porous, or semi-porous.

The internal void space defined by the membrane can include at least one gas. The gas can have a low solubility in water and be, for example, hexafluoro acetone; isopropyl acetylene; allene; tetrafluoroallene; boron trifluoride; 1,2-butadiene; 1,3-butadiene; 1,3-butadiene; 1,2,3-trichloro, 2-fluoro-1,3-butadiene; 2-methyl,1,3 butadiene; hexafluoro-1,3-butadiene; butadiyne; 1-fluoro-butane; 2-methyl-butane; decafluoro butane; 1-butene; 2-butene; 2-methy-1-butene; 3-methyl-1-butene; perfluoro-1-butene; perfluoro-1-butene; perfluoro-2-butene; 1,4-phenyl-3-butene-2-one; 2-methyl-1-butene-3-yne; butyl nitrate; 1-butyne; 2-butyne; 2-chloro-1,1,1,4,4,4-hexafluoro-butyne; 3-methyl-1-butyne; perfluoro-2-butyne; 2-bromo-butyraldehyde; carbonyl sulfide; crotononitrile; cyclobutane; methyl-cyclobutane; octafluoro-cyclobutane; perfluoro-cyclobutene; 3-chloro-cyclopentene; perfluoro ethane; perfluoro propane; perfluoro butane; perfluoro pentane; perfluoro hexane; cyclopropane; 1,2-dimethyl-cyclopropane; 1,1-dimethyl cyclopropane; 1,2-dimethyl cyclopropane; ethyl cyclopropane; methyl cyclopropane; diacetylene; 3-ethyl-3-methyl diaziridine; 1,1,1-trifluorodiazoethane; dimethyl amine; hexafluorodimethyl amine; dimethylethylamine; -bis-(Dimethyl phosphine)amine; 2,3-dimethyl-2-norbornane; perfluorodimethylamine; dimethyloxonium chloride; 1,3-dioxolane-2-one; perfluorocarbons such as and not limited to 4-methyl,1,1,1,2-tetrafluoro ethane; 1,1,1-trifluoroethane; 1,1,2,2-tetrafluoroethane; 1,1,2-trichloro-1,2,2-trifluoroethane; 1,1 dichloroethane; 1,1-dichloro-1,2,2,2-tetrafluoro ethane; 1,2-difluoro ethane; 1-chloro-1,1,2,2,2-pentafluoro ethane; 2-chloro, 1,1-difluoroethane; 1-chloro-1,1,2,2-tetrafluoro ethane; 2-chloro, 1,1-difluoroethane; chloroethane; chloropentafluoro ethane; dichlorotrifluoroethane; fluoro-ethane; hexafluoro-ethane; nitro-pentafluoro ethane; nitroso-pentafluoro ethane; perfluoro ethane; perfluoro ethylamine; ethyl vinyl ether; 1,1-dichloro ethylene; 1,1-dichloro-1,2-difluoro ethylene; 1,2-difluoro ethylene; Methane; Methane-sulfonyl chloride-trifluoro; Methanesulfonyl fluoride-trifluoro; Methane-(pentafluorothio)trifluoro; Methane-bromo difluoro nitroso; Methane-bromo fluoro; Methane-bromo chloro-fluoro; Methanebromo-trifluoro; Methane-chloro difluoro nitro; Methane-chloro dinitro; Methanechloro fluoro; Methane-chloro trifluoro; Methane-chloro-difluoro; Methane dibromo difluoro; Methane-dichloro difluoro; Methane-dichloro-fluoro; Methanedifluoro; Methane-difluoro-iodo; Methane-disilano; Methane-fluoro; Methaneiodo; Methane-iodo-trifluoro; Methane-nitro-trifluoro; Methane-nitroso-trifluoro; Methane-tetrafluoro; Methane-trichlorofluoro; Methane-trifluoro; Methanesulfenylchloride-trifluoro; 2-Methyl butane; Methyl ether; Methyl isopropyl ether; Methyl lactate; Methyl nitrite; Methyl sulfide; Methyl vinyl ether; Neon; Neopentane; Nitrogen (N.sub.2); Nitrous oxide; 1,2,3-Nonadecane tricarboxylic acid-2-hydroxytrimethylester; 1-Nonene-3-yne; Oxygen (O.sub.2); 1, 4-Pentadiene; n-Pentane; Pentane-perfluoro; 2-Pentanone-4-amino-4-methyl; 1-Pentene; 2-Pentene [cis]; 2-Pentene (trans); 1-Pentene-3-bromo; 1-Pentene-perfluoro; Phthalic acid-tetrachloro; Piperidine-2,3,6-trimethyl; Propane, Propane-1,1,1,2,2,3-hexafluoro; Propane-1,2-epoxy; Propane-2,2 difluoro; Propane 2-amino, Propane-2-chloro; Propane-heptafluoro-1-nitro; Propane-heptafluoro-1-nitroso; Propane-perfluoro; Propene; Propyl-1,1,1,2,3,3-hexafluoro-2,3 dichloro; Propylene-1-chloro; Propylenechloro-(trans); Propylene-2-chloro; Propylene-3-fluoro; Propylene-perfluoro; Propyne; Propyne-3,3,3-trifluoro; Styrene-3-fluoro; Sulfur hexafluoride; Sulfur (di)-decafluoro(S2F10); Toluene-2,4-diamino; Trifluoroacetonitrile; Trifluoromethyl peroxide; Trifluoromethyl sulfide; Tungsten hexafluoride; Vinyl acetylene; Vinyl ether; Xenon; Nitrogen; air; and other ambient gases.

In some embodiments, the gas is a perfluorocarbon. Perfluorocarbons can include fluorine gas, perfluoromethane, perfluoroethane, perfluoropropane, perfluorobutane, perfluoropentane, perfluorohexane.

In some embodiments, the nanobubbles can include a linker to link a targeting moiety to the membrane of each nanobubble. The linker can be of any suitable length and contain any suitable number of atoms and/or subunits. The linker can include one or combination of chemical and/or biological moieties. Examples of chemical moieties can include alkyl groups, methylene carbon chains, ether, polyether, alkyl amide linkers, alkenyl chains, alkynyl chains, disulfide groups, and polymers, such as poly(ethylene glycol) (PEG), functionalized PEG, PEG-chelant polymers, dendritic polymers, and combinations thereof. Examples of biological moieties can include peptides, modified peptides, streptavidin-biotin or avidin-biotin, polyaminoacids (e.g., polylysine), polysaccharides, glycosaminoglycans, oligonucleotides, phospholipid derivatives, and combinations thereof.

The nanobubbles can also include other materials, such as liquids, oils, bioactive agents, diagnostic agents, therapeutic agents, photoacoustic agents (e.g., sudan black), and/or nanoparticles (e.g., iron oxide). The materials can be encapsulated by the membrane and/or linked or conjugated to the membrane.

The targeting moiety can specifically bind to a cell surface molecule of an immune cell and is capable of targeting and/or adhering the nanobubble to the immune cell for internalization or intracellular uptake by the immune cells. In some embodiments, the targeting moiety can comprise any molecule, or complex of molecules, which is/are capable of interacting with a cell surface or extracellular molecule or biomarker of the cell. The cell surface molecule can include, for example, a cellular protease, a kinase, a protein, a cell surface receptor, a lipid, and/or fatty acid, such as NKG2D, CD45 and CD16/Fc protein.

In certain embodiments, the targeting moiety specifically binds the cell surface molecule of the immune cell. As used herein, a first molecule “specifically binds” to a second molecule if it binds to or associates with the second molecule with an affinity or Ka (that is, an equilibrium association constant of a particular binding interaction with units of 1/M) of, for example, greater than or equal to about 10⁵ M⁻¹. In certain embodiments, the first molecule binds to the second molecule with a Ka greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (KD) of a particular binding interaction with units of M (e.g., 10⁻⁵ M to 10⁻¹³ M, or less). In certain aspects, specific binding means binding to the target molecule with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the first molecule for the target can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

In some embodiments, the targeting moiety can include, but is not limited to, synthetic compounds, natural compounds or products, macromolecular entities, bioengineered molecules (e.g., polypeptides, lipids, polynucleotides, antibodies, antibody fragments), and small entities (e.g., small molecules, neurotransmitters, substrates, ligands, hormones and elemental compounds).

In one example, the targeting moiety can comprise an antibody, such as a monoclonal antibody, a polyclonal antibody, or a humanized antibody, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent targeting moieties including without limitation: monospecific or bispecific antibodies, such as disulfide stabilized Fv fragments, scFv tandems ((scFv)2 fragments), diabodies, tribodies or tetrabodies, which typically are covalently linked or otherwise stabilized (i.e., leucine zipper or helix stabilized) scFv fragments; and receptor molecules, which naturally interact with a desired target molecule.

Preparation of antibodies may be accomplished by any number of well-known methods for generating antibodies. These methods typically include the step of immunization of animals, typically mice, with a desired immunogen (e.g., a desired target molecule or fragment thereof). Once the mice have been immunized and boosted one or more times with the desired immunogen(s), antibody-producing hybridomas may be prepared and screened according to well-known methods. See, for example, Kuby, Janis, Immunology, Third Edition, pp. 131-139, W.H. Freeman & Co. (1997), for a general overview of monoclonal antibody production, that portion of which is incorporated herein by reference.

The targeting moiety need not originate from a biological source. The targeting moiety may, for example, be screened from a combinatorial library of synthetic peptides. One such method is described in U.S. Pat. No. 5,948,635, incorporated herein by reference, which describes the production of phagemid libraries having random amino acid insertions in the pIII gene of M13. This phage may be clonally amplified by affinity selection.

In other embodiments, the targeting moiety may be modified to make them more resistant to cleavage by proteases. For example, the stability of a targeting moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of targeting moiety may be of the D configuration. The switch from L to D amino acids neutralizes the digestion capabilities of many of the ubiquitous peptidases found in the digestive tract. Alternatively, enhanced stability of a targeting moiety comprising a peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of a targeting moiety may be achieved by intercalating one or more dextrorotatory amino acids (such as, dextrorotatory phenylalanine or dextrorotatory tryptophan) between the amino acids of targeting moiety. In exemplary embodiments, such modifications increase the protease resistance of a targeting moiety without affecting the activity or specificity of the interaction with a desired target molecule.

In certain embodiments, a targeting moiety as described herein may comprise a homing peptide, which selectively directs the nanobubble to an immune cell. Phage display technology provides a means for expressing a diverse population of random or selectively randomized peptides. Various methods of phage display and methods for producing diverse populations of peptides are well known in the art. For example, methods for preparing diverse populations of binding domains on the surface of a phage have been described in U.S. Pat. No. 5,223,409. In particular, phage vectors useful for producing a phage display library as well as methods for selecting potential binding domains and producing randomly or selectively mutated binding domains are also provided in U.S. Pat. No. 5,223,409. Similarly, methods of producing phage peptide display libraries, including vectors and methods of diversifying the population of peptides that are expressed, are also described in Smith et al., 1993, Meth. Enzymol., 217:228-257, Scott et al., Science, 249:386-390, and two PCT publications WO 91/07141 and WO 91/07149. Phage display technology can be particularly powerful when used, for example, with a codon based mutagenesis method, which can be used to produce random peptides or randomly or desirably biased peptides (see, e.g., U.S. Pat. No. 5,264,563). These or other well-known methods can be used to produce a phage display library, which can be subjected to the in vivo phage display method in order to identify a peptide that homes to one or a few selected tissues.

In vitro screening of phage libraries has previously been used to identify peptides that bind to antibodies or cell surface receptors (see, e.g., Smith, et al., 1993, Meth. Enzymol., 217:228-257). For example, in vitro screening of phage peptide display libraries has been used to identify novel peptides that specifically bind to integrin adhesion receptors (see, e.g., Koivunen et al., 1994, J. Cell Biol. 124:373-380), and to the human urokinase receptor (Goodson, et al., 1994, Proc. Natl. Acad. Sci., USA 91:7129-7133).

In certain embodiments, the targeting moiety may comprise a receptor molecule, including, for example, receptors, which naturally recognize a specific desired molecule of an immune cell. Such receptor molecules include receptors that have been modified to increase their specificity of interaction with a target molecule, receptors that have been modified to interact with a desired target molecule not naturally recognized by the receptor, and fragments of such receptors.

In other embodiments, the targeting moiety may comprise a ligand molecule, including, for example, ligands which naturally recognize a specific desired receptor of an immune cell. Such ligand molecules include ligands that have been modified to increase their specificity of interaction with a target receptor, ligands that have been modified to interact with a desired receptor not naturally recognized by the ligand, and fragments of such ligands.

In still other embodiments, the targeting moiety may comprise an aptamer. Aptamers are oligonucleotides that are selected to bind specifically to a desired molecular structure of the immune cell. Aptamers typically are the products of an affinity selection process similar to the affinity selection of phage display (also known as in vitro molecular evolution). The process involves performing several tandem iterations of affinity separation, e.g., using a solid support to which the diseased immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind target molecules. Aptamers typically are RNA, but may be DNA or analogs or derivatives thereof, such as, without limitation, peptide nucleic acids (PNAs) and phosphorothioate nucleic acids.

In yet other embodiments, the targeting moiety may be a peptidomimetic. By employing, for example, scanning mutagenesis to map the amino acid residues of a protein, which is involved in binding other proteins, peptidomimetic compounds can be generated that mimic those residues, which facilitate the interaction. Such mimetics may then be used as a targeting moiety to deliver the nanobubble to a target cell. For instance, non-hydrolyzable peptide analogs of such resides can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al., 1986, J Med Chem 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., 1985, Tetrahedron Lett 26:647; and Sato et al., 1986, J Chem Soc Perkin Trans 1:1231), and β-aminoalcohols (Gordon et al., 1985, Biochem Biophys Res Cummun 126:419; and Dann et al., 1986, Biochem Biophys Res Commun 134:71).

In some embodiments, the nanobubbles can be formed by dissolving at least one lipid and a lipid linked to a targeting moiety in propylene glycol. For example, a nanobubble can be prepared by dissolving 1,2-dibehenoyl-sn-glycero-3-phosphocholine (DBPC, Avanti Polar Lipids Inc., Pelham, AL), 1,2-Dipalmitoyl-sn-glycero-3-Phosphate; DPPA, 1,2-dipalmitoyl-sn-glycero-3-phosphor ethanolamine; DPPE (Corden Pharma, Switzerland), and 1,2-distearoyl-sn-glycero-3-phosphoethanolamine-N-[methoxy (polyethylene glycol)-2000](ammonium salt) (DSPE-mPEG 2000, Laysan Lipids, Arab, AL) in propylene glycol to produce a lipid-propylene glycol solution. It will be appreciated that other materials can be dissolved in the propylene glycol, such as proteins, carbohydrates, polymers, surfactants, and/or other membrane stabilizing materials.

After producing the lipid-propylene glycol solution, a glycerol and phosphate buffered solution (PBS) solution can be added to lipid-propylene glycol solution and the resulting solution can be mixed by, for example, sonication. The mixed solution can be transferred to a vial. The air can removed from the sealed vial containing the hydrated lipid solution and replaced with a gas, such as octafluoropropane, until the vial pressure equalized. The resultant solution can then be shaken or stirred for a time (e.g., about 45 seconds) sufficient to form the nanobubbles. In one example, a lipid-propylene glycol solution comprising DBPC/DPPA/DPPE/DSPE-PEG dissolved in propylene glycol can be contacted with a hydration PBS/glycerol solution, placed in a vial, and then placed in an incubator-shaker at about 37° C. and at about 120 rpm for about 60 minutes. In some embodiments, the resultant solution containing the nanobubbles can be freeze dried and reconstituted for storage and shipping or frozen and thawed before use.

In some embodiments, the sizes of nanobubbles can be adjusted, if desired, by a variety of procedures including extrusion, filtration, sonication, homogenization, employing a laminar stream of a core of liquid introduced into an immiscible sheath of liquid, extrusion under pressure through pores of defined size, and similar methods. The foregoing techniques, as well as others, are discussed, for example, in U.S. Pat. No. 4,728,578; U.K. Patent Application GB 2193095 A; U.S. Pat. Nos. 4,728,575; 4,737,323; International Application PCT/US85/01161; Mayer et al., Biochimica et Biophysica Acta, Vol. 858, pp. 161-168 (1986); Hope et al., Biochimica et Biophysica Acta, Vol. 812, pp. 55-65 (1985); U.S. Pat. No. 4,533,254; Mayhew et al., Methods in Enzymology, Vol. 149, pp. 64-77 (1987); Mayhew et al., Biochimica et Biophysica Acta, Vol 755, pp. 169-74 (1984); Cheng et al, Investigative Radiology, Vol. 22, pp. 47-55 (1987); PCT/US89/05040, U.S. Pat. Nos. 4,162,282; 4,310,505; 4,921,706; and Liposome Technology, Gregoriadis, G., ed., Vol. I, pp. 29-31, 51-67 and 79-10⁸ (CRC Press Inc., Boca Raton, Fla. 1984). The disclosures of each of the foregoing patents, publications and patent applications are incorporated by reference herein, in their entirety.

Filter pore sizes are selected for sizing as well as to remove any potential contaminants. The filter pore size may be between 10 nm and 1 m, more preferably between 30 nm and 1 m, and even more preferably between 100 nm and 1 m. Two or more filters may be stacked in a series to maximize the effectiveness of filtration. Useful materials for formation of the filters include polymers such as polysulfonate, polycarbonate, and polyvinylidene chloride. In addition, glass, ceramics, and metal filters may also be utilized. Additionally, wire, polymer, or ceramic meshes may also be utilized. Filtration may either be utilized. Filtration may be performed as part of the manufacturing process or during administration through an in-line filter.

In some embodiments, each of the nanobubbles can have a size that facilitates internalization of the cell targeted nanobubbles by the immune cells. For example, each of the nanobubbles can have a size (diameter) of about 30 nm to about 600 nm or about 100 nm to about 500 nm (e.g., about 300 nm), depending upon the particular lipids, edge activator, and membrane stiffener as well as the method used to form the nanobubble (described in greater detail below).

For storage prior to use, the nanobubbles may be suspended in an aqueous solution, such as a saline solution (for example, a phosphate buffered saline solution), or simply water, and stored preferably at a temperature of between about 2° C. and about 10° C., preferably at about 4° C. Preferably, the water is sterile. Most preferably, the nanobubbles are stored in an isotonic saline solution, although, if desired, the saline solution may be a hypotonic saline solution (e.g., about 0.3 to about 0.5% NaCl). The solution also may be buffered, if desired, to provide a pH range of about pH 5 to about pH 7.4. Suitable buffers for use in the storage media include, but are not limited to, acetate, citrate, phosphate and bicarbonate.

Bacteriostatic agents may also be included with the nanobubbles to prevent bacterial degradation on storage. Suitable bacteriostatic agents include but are not limited to benzalkonium chloride, benzethonium chloride, benzoic acid, benzyl alcohol, butylparaben, cetylpyridinium chloride, chlorobutanol, chlorocresol, methylparaben, phenol, potassium benzoate, potassium sorbate, sodium benzoate and sorbic acid. One or more antioxidants may further be included with the gaseous precursor-filled liposomes to prevent oxidation of the lipid. Suitable antioxidants include tocopherol, ascorbic acid and ascorbyl palmitate.

In some embodiments, the immune cells can be labeled with the nanobubbles by mixing an enriched population of immune cells with a plurality of nanobubbles and co-culturing or incubating the mixture of immune cells and the plurality of nanobubbles until at least about 10%, at least about 20%, at least about 30%, at least about 40%, at least about 50%, at least about 60%, at least about 70%, or more of the immune cells uptake or internalize the nanobubbles to provide intracellular labeled immune cells. Labeling at least about 10% of the immune cells with the nanobubbles can provide a contrast to tissue (CTR) ratio of greater than about 2 dB, for example, about 4 dB to about 15 dB or about 5 dB

In some embodiments, at least about 500, at least about 1000, at least about 2000, at least about 3000, at least about 4000, at least about 5000, at least about 6000, at least about 7000, at least about 8000, at least about 9000, at least about 10,000, at least about 25,000, at least about 50,000 nanobubbles, or at least about 100,000 nanobubbles per immune cell can be co-cultured with the immune cells at a temperature of, for example, about 37° C. and 5% CO₂ for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, or more to promote uptake of the nanobubbles by the immune cells.

Following incubation, unbound nanobubbles or nanobubbles not internalized by the immune cells can be removed from the mixture by spinning the mixture, for example, at about 1000 or 2000 rpm, and washing the immune cells with, for example, PBS. The enriched population of immune cells intracellularly labeled with nanobubbles can then be suspended in PBS and stored at reduced temperature until use.

In some embodiments, acoustic pulses can be applied to the mixture immune cells and nanobubbles during co-culturing or incubation to stimulate nanobubble endocytosis by the immune cells. A combination of acoustic vibrations and nanobubble oscillations can help drive small nanobubbles into cells without active targeting approaches.

In other embodiments, the nanobubbles mixed with immune cells can include a targeting moieties that targets the nanobubbles to the immune cells and promotes uptake of the nanobubbles by the immune cells. Targeting the nanobubbles to the immune cells in culture can have the advantage of providing a highly efficient strategy to drive nanobubble uptake into the immune cells. The nanobubbles can be labeled with antibodies to immune cell surface receptors or protein, such as NKG2D, CD45 and CD16/Fc protein. Nanobubbles can be functionalized with the targeting moiety using a linker, such as standard EDC/NHS conjugation chemistry and purified by size exclusion chromatography.

The enriched population of immune cells intracellularly labeled with the nanobubbles can be included in a composition, such as a pharmaceutical composition, for immunotherapy, adoptive immunotherapy, and/or treating cancer or an infectious disease. Adoptive immunotherapy using an enriched population of immune cells, such as NK cells and T-cells, has shown clinical promise against a wide variety of tumor types, including both solid tumors and blood cancers. The therapeutic approaches described herein can be used as treatment of virtually all types of cancers and pre-cancers (e.g., Myelodysplastic syndrome), including but not limited to carcinomas, sarcomas, melanomas, lymphomas, and leukemias, and having places of origin including but not limited to colon, prostate, brain, breast, liver, lung, pancreatic, bone, ovarian, skin, pancreatic, blood and others. The methods disclosed herein are contemplated for treatment of both metastatic cancers as well as primary tumor sites.

Adoptive immunotherapy has also been employed to treat viral infections, including Cytomegalovirus, human immunodeficiency virus (HIV), hepatitis C virus (HCV), influenza virus, pox virus, and herpes viruses, with highly positive clinical outcomes. The methods disclosed herein are contemplated for treatment of viral infections. In a preferred embodiment, the methods disclosed herein can be used to treat HIV infections.

In some embodiments, the composition including the enriched population of immune cells intracellularly labeled with the nanobubbles can also include a pharmaceutically acceptable carrier. With respect to pharmaceutical compositions, the carrier can be any of those conventionally used for the administration of cells. Such pharmaceutically acceptable carriers are well-known to those skilled in the art and are readily available to the public. It is preferred that the pharmaceutically acceptable carrier be one which has no detrimental side effects or toxicity under the conditions of use.

The compositions can be prepared in unit dosage forms for administration to a subject. The amount and timing of administration are at the discretion of the treating clinician to achieve the desired outcome. The compositions can be formulated for systemic (such as intravenous) or local (such as intra-tumor) administration. In one example, an enriched population of immune cells intracellularly labeled with the nanobubbles is formulated for parenteral administration, such as intravenous administration. Compositions including an enriched population of immune cells intracellularly labeled with the nanobubbles as disclosed herein can be used, for example, for the treatment a tumor.

The compositions for administration can include a solution of the enriched population of immune cells intracellularly labeled with the nanobubbles provided in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions, such as pH adjusting and buffering agents, toxicity adjusting agents, adjuvant agents, and the like, for example, sodium acetate, sodium chloride, potassium chloride, calcium chloride, sodium lactate and the like. The concentration of the enriched population of immune cells intracellularly labeled with the nanobubbles in these formulations can vary widely, and will be selected primarily based on fluid volumes, viscosities, body weight and the like in accordance with the particular mode of administration selected and the subject's needs. Actual methods of preparing such dosage forms for use in in gene therapy, immunotherapy and/or cell therapy are known, or will be apparent, to those skilled in the art.

In one example, the enriched population of immune cells intracellularly labeled with the nanobubbles can be added to an infusion bag containing 0.9% sodium chloride, USP, and in some cases administered at a dosage of from 0.5 to 15 mg/kg of body weight. An enriched population of immune cells intracellularly labeled with the nanobubbles can be administered by slow infusion, rather than in an intravenous push or bolus. In one example, a higher loading dose is administered, with subsequent, maintenance doses being administered at a lower level.

In some embodiments, the enriched population of immune cells intracellularly labeled with the nanobubbles are locally administered to a subject to improve NK cell or T cell trafficking to the targeted site, such as a solid tumor site of the subject. In some embodiments, local administration to a tumor cite can include intratumoral, intracranial, intrapleural and hepatic artery delivery.

In some embodiments, the enriched population of immune cells intracellularly labeled with the nanobubbles can be loaded on or in a biopolymer device allowing for NK cell or T cell proliferation. The immune cell loaded device can then be implanted directly to a targeted site in a subject in order to improve trafficking and tumor infiltration.

The dose, e.g., number of immune cells intracellularly labeled with the nanobubbles administered should be sufficient to effect, e.g., a therapeutic or prophylactic response, in the subject or animal over a reasonable time frame. For example, the number of immune cells intracellularly labeled with the nanobubbles should be sufficient to treat or prevent cancer in a period of from about 2 hours or longer, e.g., 12 to 24 or more hours, from the time of administration. In certain embodiments, the time period could be even longer. The number of cells of the enriched population of immune cells intracellularly labeled with the nanobubbles will be determined by, e.g., the efficacy of enriched population of immune cells intracellularly labeled with the nanobubbles and the condition of the animal (e.g., human), as well as the body weight of the animal (e.g., human) to be treated.

The number of immune cells administered to the subject also will be determined by the existence, nature and extent of any adverse side effects that might accompany the administration of an enriched population of immune cells intracellularly labeled with the nanobubbles. Typically, the attending physician will decide the number of the enriched population of immune cells intracellularly labeled with the nanobubbles with which to treat each individual patient, taking into consideration a variety of factors, such as age, body weight, general health, diet, sex, route of administration, and the severity of the condition being treated. By way of example and not intending to limit the invention, the number of immune cells in the enriched population of immune cells intracellularly labeled with the nanobubbles can be about at least about 1 million immune cells per infusion, at least about 2 million immune cells per infusion, at least about 3 million immune cells per infusion, at least about 4 million immune cells per infusion, at least about 5 million immune cells per infusion, at least about 10 million immune cells, 10×10⁴ to about 10×10¹¹ cells per infusion, about 10×10⁵ cells to about 10×10⁹ cells per infusion, or 10×10⁷ to about 10×10⁹ cells per infusion.

The enriched population of immune cells intracellularly labeled with the nanobubbles can be administered to patients via infusion. Preferably, cell transfers occur biweekly, once weekly, once every two weeks, once every three weeks, once every four weeks, once every five weeks, once every six weeks, or on an as-needed basis. In a preferred embodiment, the enriched population of immune cells intracellularly labeled with the nanobubbles infusions will be administered in two week intervals.

For purposes of the inventive methods, the administered enriched population of immune cells intracellularly labeled with the nanobubbles can include cells that are allogeneic or autologous to the host or subject. Preferably, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive NK cell therapy or T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

With respect to the methods, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia (CLL), hairy cell leukemia, acute lymphocytic leukemia (ALL), acute myeloid leukemia (AML), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer.

In some embodiments, a composition comprising the enriched population of immune cells intracellularly labeled with the nanobubbles can be administered in combination with an agent that increases the anti-cancer effects of the composition. The enriched population of immune cells intracellularly labeled with the nanobubbles may be co-administered to a subject with any cancer treatment known in the art.

In one embodiment, the subject is treated with the enriched population of immune cells intracellularly labeled with the nanobubbles and an antiproliferative agent. Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

In one embodiment, the subject is treated with the enriched population of immune cells intracellularly labeled with the nanobubbles and a chemotherapeutic agent. Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

In one embodiment, the subject is treated with the enriched population of immune cells intracellularly labeled with the nanobubbles and another anti-tumor agent, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine. Anti-angiogenic agents are well known to those of skill in the art. Suitable anti-angiogenic agents for use in the methods and reprogrammed T cells of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

It will be appreciated that other disease or conditions can be treated with the enriched population of immune cells intracellularly labeled with nanobubbles. Such diseases or conditions include an infectious disease or condition, such as, but not limited to, viral, retroviral, bacterial, and protozoan infections, immunodeficiency, Cytomegalovirus (CMV), Epstein-Barr virus (EBY), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or a disease or condition associated with transplant.

Once the enriched population of immune cells intracellularly labeled with the nanobubbles is administered to a mammal (e.g., a human), the immune cells can be monitored or tracked in a region of interest (ROI) in a subject using ultrasound contrast imaging to determine localization and/or distribution of the immune cells in the ROI and potentially effectiveness of the immune cells in treating the disease or disorder. At least one image of the ROI can be generated using ultrasound from a time, for example, 0 to about 48 hours or 0 to about 24 hours after administration.

The image can be generated by applying ultrasound energy to the ROI and acquiring ultrasound data for the ROI of interest in response to the applied energy. The ROI can include neoplastic tissue, such as cancer tissue or tumors including solid carcinomas, sarcomas or lymphomas, and/or an aggregate of neoplastic cells, in the subject. The at least one image of the ROI can be indicative of whether the immune cells of the immunotherapy composition are in the ROI. Optionally, a second dose of the enriched population of immune cells intracellularly labeled with the nanobubbles can be administered to the subject if the amount of immune cells in the ROI after the first dose is less than a control amount.

In diagnostic ultrasound, which may be used to monitor the location of the immune cell intracellularly labeled with the nanobubbles, an ultrasound transducer applies one or several pulses of an acoustic signal to the ROI and receives the reflected signal between pulses. The limited number of pulses used in diagnostic ultrasound limits the effective energy which is delivered to the tissue which is being imaged.

Either fixed frequency or modulated frequency ultrasound may be used. Fixed frequency is defined wherein the frequency of the sound wave is constant over time. A modulated frequency is one in which the wave frequency changes over time, for example, from high to low (PRICH) or from low to high (CHIRP). For example, a PRICH pulse with an initial frequency of 10 MHz of sonic energy is swept to 1 MHz with increasing power from 1 to 5 watts.

In some embodiments, the frequency of the ultrasound used may vary from about 0.2 kHz to about 50 MHz, for example, about 0.75 and about 30 megahertz. Commonly used diagnostic frequencies of about 3 to about 20 megahertz may also be used. For smaller, e.g., below 300 nm diameter, higher frequencies of sound may be used as these smaller nanobubbles can absorb sonic energy more effectively at higher frequencies of sound. When very high frequencies are used, e.g., over 10 megahertz, the sonic energy will generally have limited depth penetration into fluids and tissues. External application may be preferred for the skin and other superficial tissues, but for deep structures, the application of sonic energy via interstitial probes or intravascular ultrasound catheters may be preferred.

In some embodiments, ultrasound contrast imaging includes applying ultrasound energy to the ROI at a duty cycle of about 1% to about 100%, an ultrasound frequency of about 0.2 kHz to about 50 MHz, an intensity of about 0.1 W/cm² to about 5 W/cm², a pressure amplitude of about 50 kPa to about 10 MPa, and a time of about 1 minute to about 30 minutes to monitor or track immune cell trafficking.

The following example is for the purpose of illustration only and is not intended to limit the scope of the claims, which are appended hereto.

### Example 1

### Preparation of Nanobubbles

Nanobubbles (NBs) (10 mg/mL) were prepared by first dissolving a mixture of lipids comprising of 1,2-dibehenoyl-sn-glyc-ero-3-phosphocholine (C22, Avanti Polar Lipids Inc., Pelham, AL), 1,2 Dipalmitoyl-sn-Glycero-3-Phosphate (DPPA, Corden Pharma, Switzerland), 1,2-dipalmitoyl-sn-glycero-3-phosphoethanolamine (DPPE, Corden Pharma, Switzerland), and 1,2-distearoyl-snglycero-3-phosphoethanolamine-N-[methoxy(polyethylene glycol)-2000] (ammonium salt) (DSPE-mPEG 2000, Laysan Lipids, Arab, AL) into propylene glycol (0.1 mL, Sigma Aldrich, Milwaukee, WI) by heating and sonicating at 80° C. until all the lipids were dissolved. Mixture of glycerol (0.1 mL, Acros Organics) and phosphate buffered saline (0.8 mL, Gibco, pH 7.4) preheated to 80° C. was added to the lipid solution. The solution was transferred to a 3 mL-headspace vial, capped with a rubber septum and aluminum seal, and sealed with a vial crimper. Air was manually removed with a 30 mL-syringe and was replaced by injecting octafluoropropane (C₃F₈, Electronic Fluorocarbons, LLC, PA) gas. The phospholipid solution was then activated by mechanical shaking with a VialMix shaker (Bristol-Myers Squibb Medical Imaging Inc., N. Billerica, MA) for 45 s. NBs were isolated from the mixture of foam and microbubbles by centrifugation at 50 rcf for 5 min with the headspace vial inverted, then 200 μL NB solution was withdrawn from a fixed distance of 5 mm from the bottom with a 21G needle.

### Example 2

### Non-Invasive Trafficking of Nanobubble Tagged CAR T-Cells Using Clinical Grade Ultrasound Methods

### In Vitro Studies

CAR T-cell were co-cultured with nanobubbles at varying concentrations (5000, 10000, and 50000 NB/cell) at different time points (2 hr, 4 hr, and 24 hr). Cells were spun, washed, suspended in RPMI media, and placed in ice. Sample injected into static/flow phantom and scanned 2 min at 12 MHz, 1 fps, 0.19 and 0.32 MI value using Toshiba clinical-grade ultrasound. CAR T-cell cytotoxicity assay was performed to determine the preserved cytotoxic function of NB tagged CAR T-cells.

### In Vivo Studies

Cells prepared similarly as mentioned above with the optimization parameters obtained from in vitro studies; 10,000 NB/cell and 2 hr incubation period. Sample injected into the tail vein of healthy NSG mice and imaging of heart/lung, liver, kidney performed immediately after infusion, 30 min, 3 hr, 6 hr, and 24 hr time points. After 24 hours, mice were sacrificed, and the organs were imaged using Maestro.

### Results

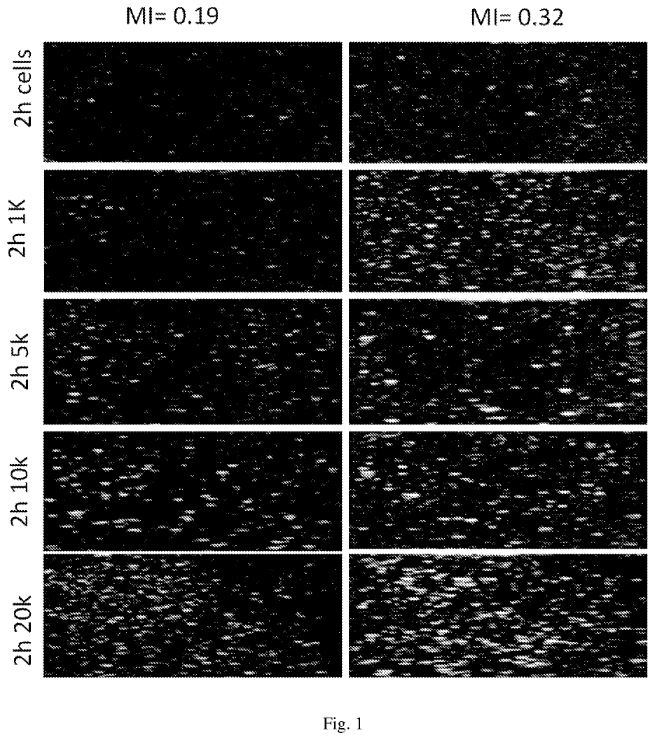

FIG. 1 illustrate ultrasound contrast (UC) images of CAR T-cells internally labeled with 1,000, 5,000, 10,000, and 20,000 nanobubbles (NBs)/cell after 2 hours incubation at an MI of 0.19 and 0.32.

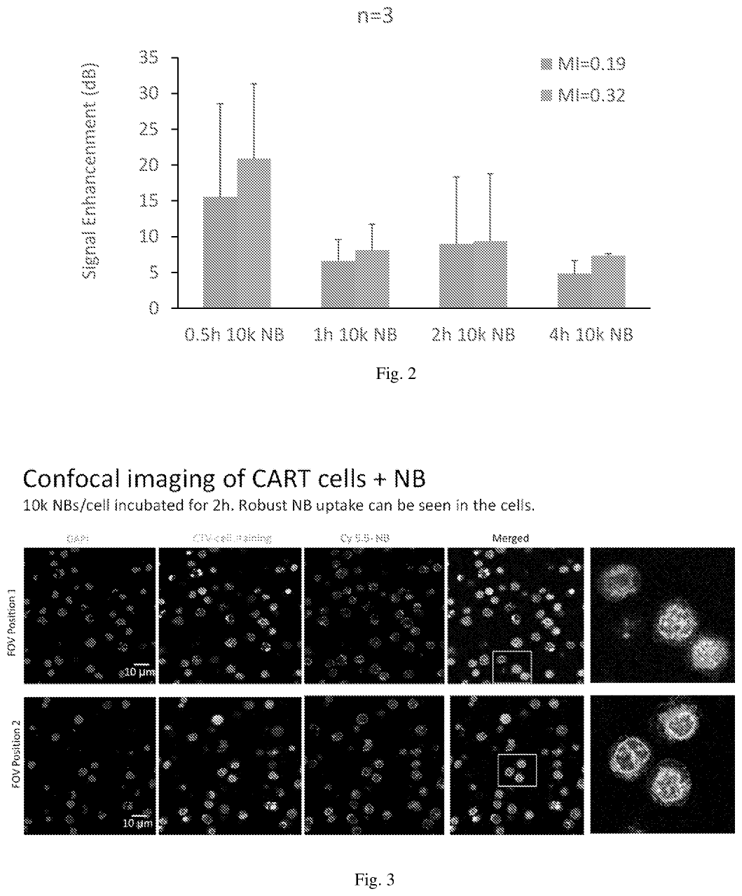

FIG. 2 illustrates a graph showing signal enhancement of CAR T-cells internally labeled with 10,000 nanobubbles (NBs)/cell after 0.5 hours, 1 hour, 2 hours, and 4 hours incubation at an MI of 0.19 and 0.32.

FIG. 3 illustrates confocal images of CAR T-cells internally labeled with 10,000 NBs/cells incubated for 2 hours. Robust uptake can be seen in the cells.

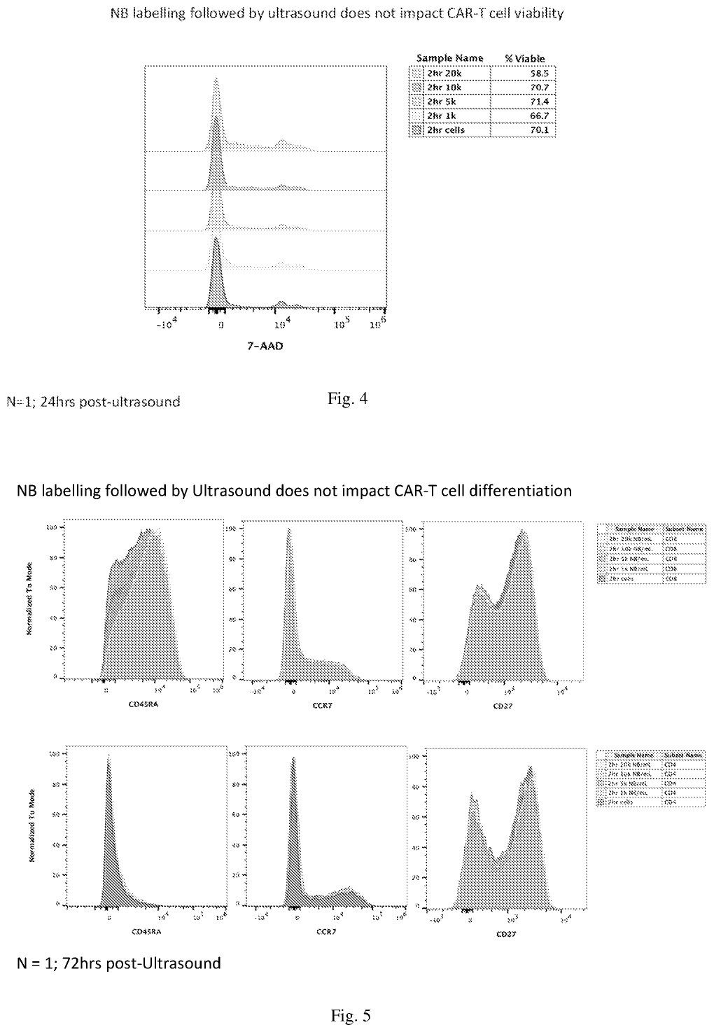

FIG. 4 illustrates plots showing cytotoxicity of CAR T-cells internally labeled with 1,000, 5,000, 10,000, and 20,000 NBs/cell 24 hours post-ultrasound. N=1; 24 hrs post-ultrasound; 2 hr NB co-culture, 4 hr Calcein AM cytotoxicity assay.

FIG. 5 illustrates plots showing NB labelling by ultrasound does not impact CAR T-cell differentiation. N=1; 72 hrs post-ultrasound.

FIG. 6 illustrates images of heart/lung of mice intravenously injected with 200 L of 20M CAR T-cells internally labeled with Cy5.5-labeled NBs 2 hours, 24 hours, 48 hours, and 72 hours post injection.

FIG. 7 illustrates images of liver of mice intravenously injected with 200 μL of 20M CAR T-cells internally labeled with Cy5.5-labeled NBs 2 hours, 24 hours, 48 hours, and 72 hours post injection.

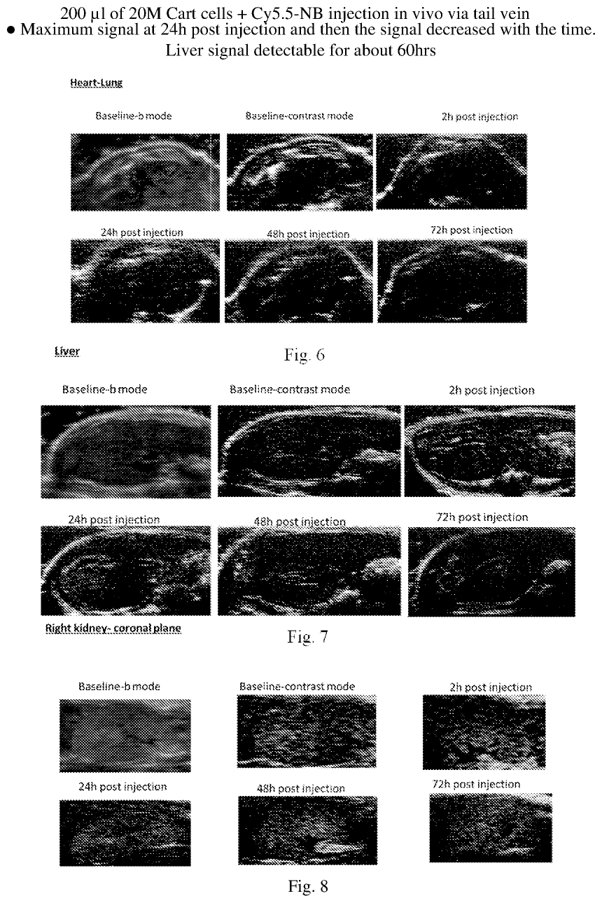

FIG. 8 illustrates images of right kidney of mice intravenously injected with 200 L of 20M CAR T-cells internally labeled with Cy5.5-labeled NBs 2 hours, 24 hours, 48 hours, and 72 hours post injection.

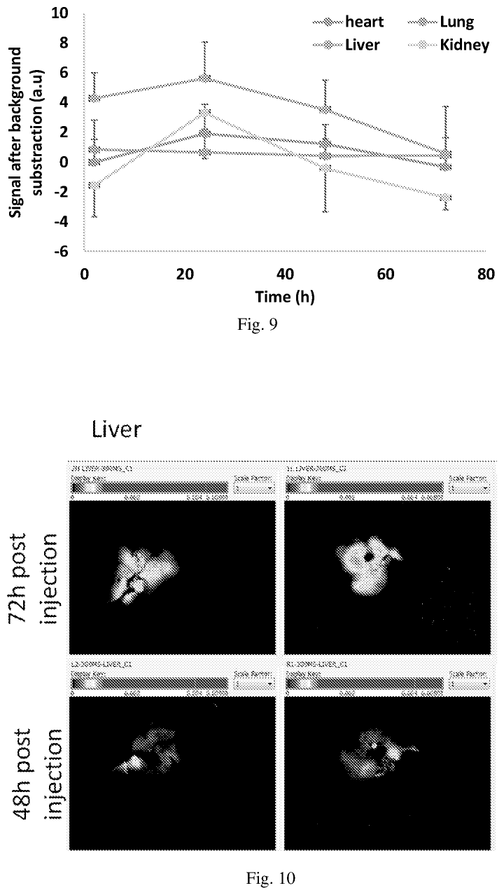

FIG. 9 illustrates plots of signal after background subtraction of heart/lung, liver, and kidney of mice intravenously injected with 200 μL of 20M CAR T-cells internally labeled with Cy5.5-labeled NBs 2 hours, 24 hours, 48 hours, and 72 hours post injection. Maximum signal 24 hours post injection. Liver signal detectable for about 60 hrs

FIG. 10 illustrates UC images of liver of mice intravenously injected with 200 L of 20M CAR T-cells internally labeled with Cy5.5-labeled NBs 48 hours and 72 hours post injection.

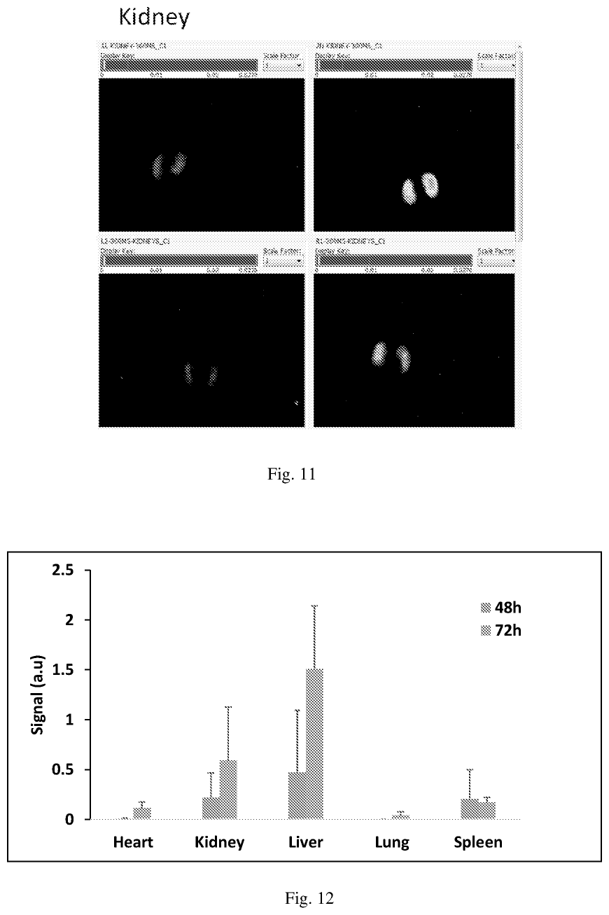

FIG. 11 illustrates UC images of right kidney of mice intravenously injected with 200 μL of 20M CAR T-cells internally labeled with Cy5.5-labeled NBs 48 hours and 72 hours post injection.

FIG. 12 illustrates plots of signal after background subtraction of heart/lung, liver, and kidney of mice intravenously injected with 200 μL of 20M CAR T-cells internally labeled with Cy5.5-labeled NBs 48 hours and 72 hours post injection.

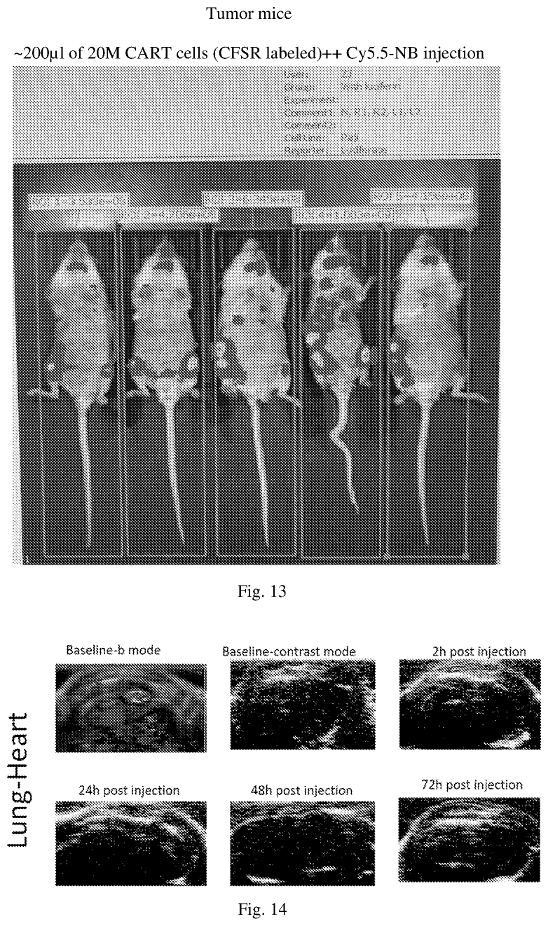

FIG. 13 illustrates images of tumor mice intravenously injected with 200 μL of 20M CAR T-cells CFSE labeled and internally labeled with Cy5.5-labeled NBs 48 hours and 72 hours post injection.

FIG. 14 illustrates images of heart/lung of tumor mice intravenously injected with 200 μL of 20M CAR T-cells internally labeled with Cy5.5-labeled NBs 2 hours, 24 hours, 48 hours, and 72 hours post injection.

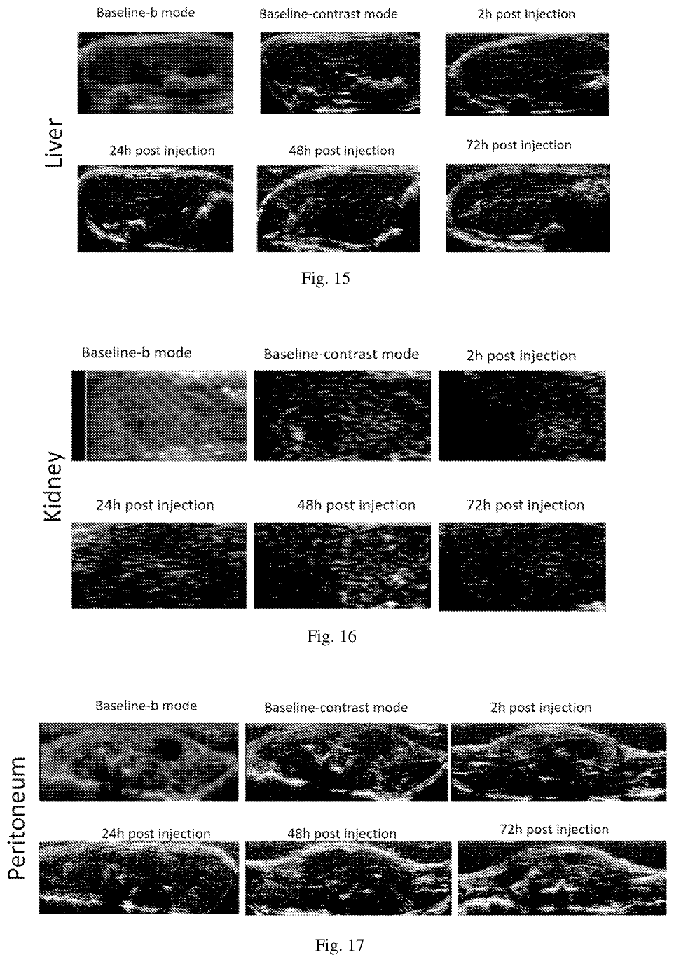

FIG. 15 illustrates images of liver of tumor mice intravenously injected with 200 L of 20M CAR T-cells internally labeled with Cy5.5-labeled NBs 2 hours, 24 hours, 48 hours, and 72 hours post injection.

FIG. 16 illustrates images of right kidney of tumor mice intravenously injected with 200 μL of 20M CAR T-cells internally labeled with Cy5.5-labeled NBs 2 hours, 24 hours, 48 hours, and 72 hours post injection.

FIG. 17 illustrates images of peritoneum of tumor mice intravenously injected with 200 μL of 20M CAR T-cells internally labeled with Cy5.5-labeled NBs 2 hours, 24 hours, 48 hours, and 72 hours post injection.

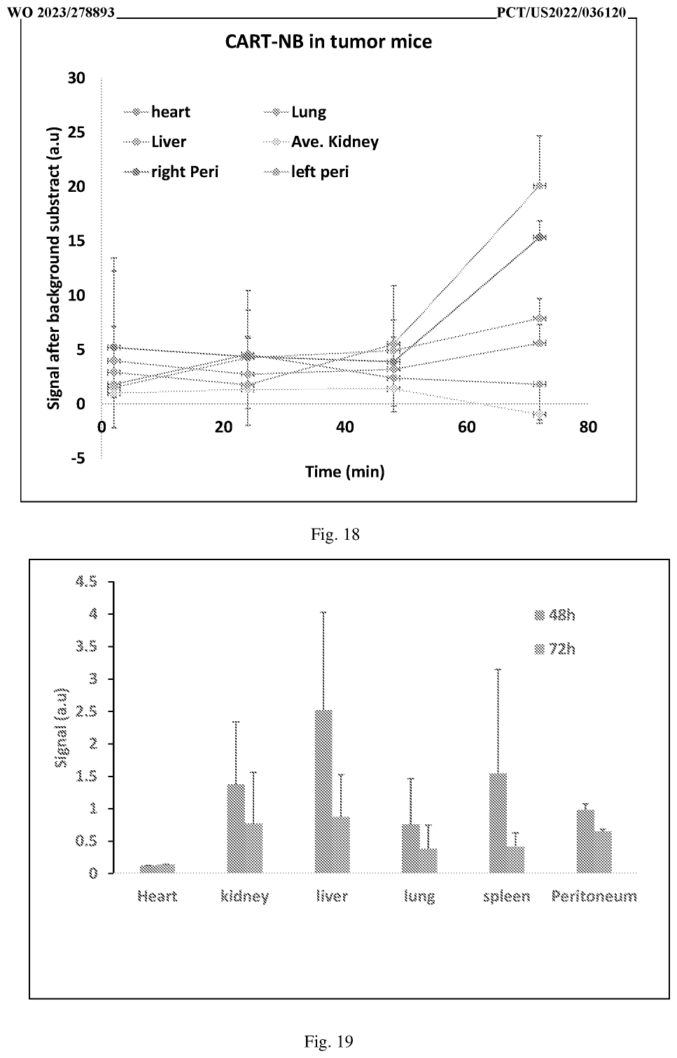

FIG. 18 illustrates plots of signal after background subtraction of heart/lung, liver, and kidney of mice intravenously injected with 200 μL of 20M CAR T-cells internally labeled with Cy5.5-labeled NBs 2 hours, 24 hours, 48 hours, and 72 hours post injection.

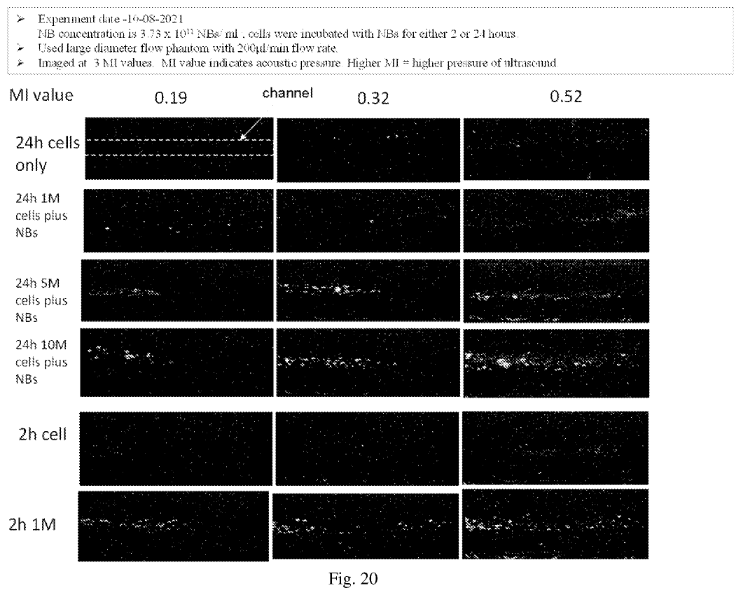

FIG. 19 illustrates a graph of detected signal of heart, kidney, liver, lung, spleen, and peritoneum of mice intravenously injected with 200 μL of 20M CAR T-cells internally labeled with Cy5.5-labeled NBs 48 hours and 72 hours post injection.

### Example 3

### Non-Invasive Trafficking of Nanobubble Tagged NK Cells Using Clinical Grade Ultrasound Methods

### In Vitro Studies

Primary NK cells from PBMCs of healthy human volunteers were isolated and expanded which were then co-cultured with nanobubbles at varying concentrations (5000, 10000, and 50000 NB/cell) at different time points (2 hr, 4 hr, and 24 hr). Cells were spun, washed, suspended in RPMI media, and placed in ice. Sample injected into static/flow phantom and scanned for about 2 min at 12 MHz, 1 fps, 0.19 and 0.32 MI value using Toshiba clinical-grade ultrasound. NK cell cytotoxicity assay was performed to determine the preserved cytotoxic function of NB tagged NK cells.

### In Vivo Studies

Cells prepared similarly as mentioned above with the optimization parameters obtained from in vitro studies; 10,000 NB/cell and 2 hr incubation period. Sample injected into the tail vein of healthy NSG mice and imaging of heart/lung, liver, kidney performed immediately after infusion, 30 min, 3 hr, 6 hr, and 24 hr time points. After 24 hours, mice were sacrificed, and the organs were imaged using Maestro.

### Results

FIG. 20 illustrates ultrasound contrast (UC) images of natural killer (NK) cells internally labeled with 1,000, 5,000, 10,000, and 20,000 nanobubbles (NBs)/cell after 2 hours and 24 hours incubation at an MI of 0.19, 0.32, and 0.52.

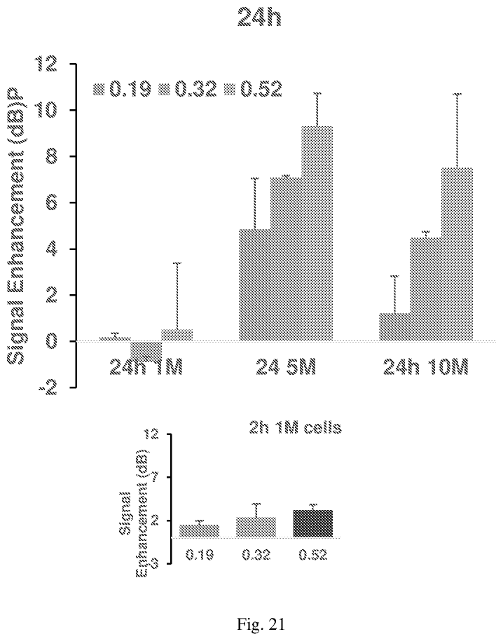

FIG. 21 illustrates a graph showing signal enhancement of 1M, 5M, and 10M NK cells internally labeled with 10,000 nanobubbles (NBs)/cell after 24 hours incubation at an MI of 0.19, 0.32, and 0.52.

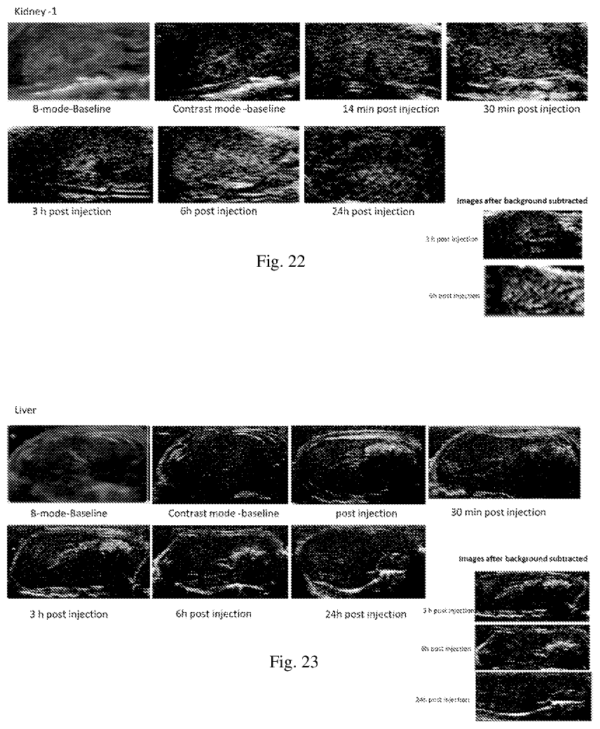

FIG. 22 illustrates US images of kidney of tumor mice intravenously injected with 200 μL of 5-10M NK-cells internally labeled with Cy5.5-labeled NBs 14 minutes, 30 minutes, 3 hours, 6 hours, and 24 hours post injection.

FIG. 23 illustrates US images of liver of tumor mice intravenously injected with 200 μL of 5-10M NK-cells internally labeled with Cy5.5-labeled NBs 5 minutes, 30 minutes, 3 hours, 6 hours, and 24 hours post injection.

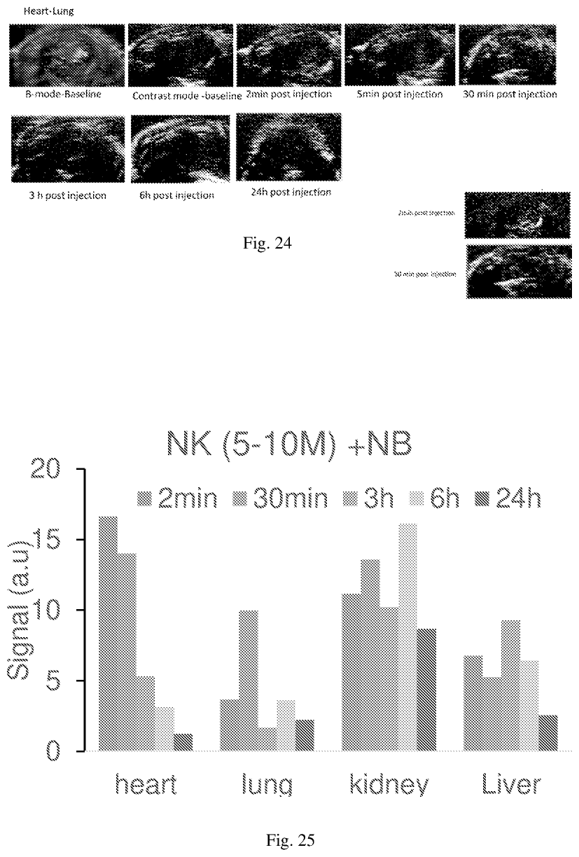

FIG. 24 illustrates US images of heart/lung of tumor mice intravenously injected with 200 μL of 5-10M NK-cells internally labeled with Cy5.5-labeled NBs 30 minutes, 3 hours, 6 hours, and 24 hours post injection.

FIG. 25 illustrates a graph of detected US signal of heart, lung, kidney, and liver of tumor mice intravenously injected with 200 μL of 5-10M NK-cells internally labeled with Cy5.5-labeled NBs 2 minutes, 30 minutes, 3 hours, 6 hours, and 24 hours post injection.

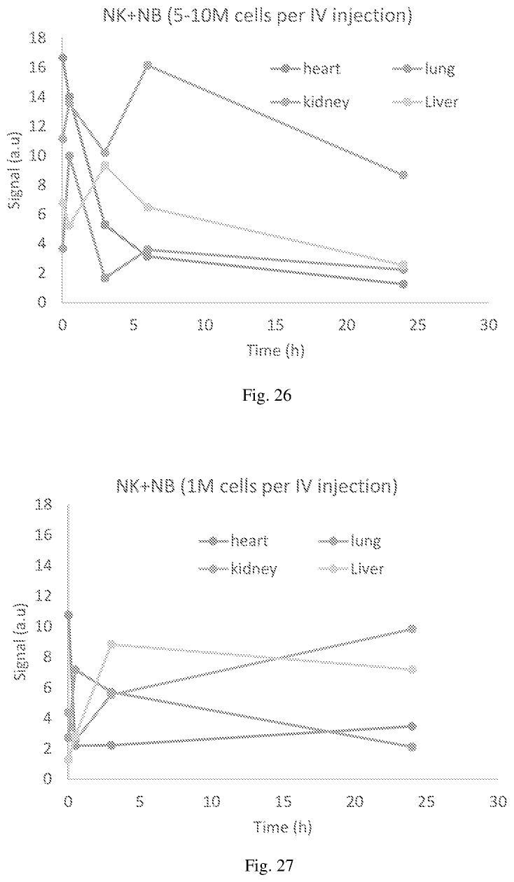

FIG. 26 illustrates plots of detected US signal of heart, lung, kidney, and liver of tumor mice intravenously injected with 200 μL of 5-10M NK-cells internally labeled with Cy5.5-labeled NBs up to 24 hours post injection.

FIG. 27 illustrates plots of detected US signal of heart, lung, kidney, and liver of tumor mice intravenously injected with 200 μL of 1M NK-cells internally labeled with Cy5.5-labeled NBs up to 24 hours post injection.

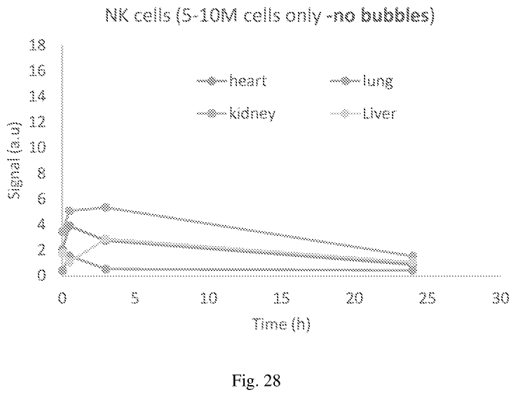

FIG. 28 illustrates plots of detected US signal of heart, lung, kidney, and liver of tumor mice intravenously injected with 200 μL of 5-10M NK-cells up to 24 hours post injection.

In vitro studies demonstrated consistent and reproducible NB signals using the ultrasound at 2 hr time point with 10000 NB/cell concentration. US signals were tested and detected both in static phantom and flow phantom (to simulate in vivo blood flow) by in vitro studies. In vivo studies in healthy mice with the injection of 5-10 million NB tagged NK cells (test) and NK cells only (control) samples demonstrated overall highest signals from test samples in contrast to control samples. The heart showed the strongest signals right after the injection, followed by the lungs 30 minutes later, the kidney 6 hours later, and the liver 3-6 hours later. Though signals in all these organs decreased with time, they were still stronger in the test than in control mice at 24 hours.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

1: An adoptive immunotherapy composition comprising:
an enriched population of immune cells, wherein, at least about 50% or more immune cells include intracellular nanobubbles.

2: The composition of claim 1, wherein at least 70% of the immune cells of the population include T-cells and/or natural killer (NK) cells that are optionally genetically modified.

3: The composition of claim 1, wherein at least 70% of the immune cells are NK cells optionally genetically modified to express one or more proteins capable of providing cytokine support.

4: The composition of claim 3, wherein the one or more proteins capable of providing cytokine support are selected from the group consisting of mbIL-15, soluble IL-15, soluble IL-21, mbIL-21, mb-IL-2, or soluble IL-2.

5: The composition of claim 3, wherein at least 70% of the immune cells are NK cells genetically modified to express one or more proteins capable of inhibiting TGFβ signaling.

6: The composition of claim 1, wherein least at 70% of the immune cells are CD4+ T-cells and/or CD8+ T-cells.

7: The composition of claim 1, wherein at least 70% of the immune cells are chimeric antigen receptor (CAR)-CD4+ T cells and/or CAR-CD8+ T cells.

8. (canceled)

9: The composition of claim 1, wherein the enriched population of cells includes an amount of intracellular nanobubbles effective to detect the enriched population of cells by ex vivo ultrasound contrast imaging upon administration of the enriched population of cells to a subject.

10: The composition of claim 1, wherein each of the immune cells that includes intracellular nanobubbles includes at least about 100 or more intracellular nanobubbles.

11: The composition of claim 1, wherein each of the nanobubbles includes a lipid membrane that defines an internal void, which includes at least one gas.

12: The composition of claim 11, wherein the lipid membrane further includes at least one of glycerol, propylene glycol, pluronic (poloxamer), alcohols or cholesterols, that change the modulus and/or interfacial tension of the bubble membrane.

13: The composition of claim 11, wherein the lipid membrane includes a mixture of phospholipids having varying acyl chain lengths.

14. (canceled)

15: The composition of claim 11, wherein the mixture of lipids includes at least about 50% by weight of dibehenoylglycerophosphocoline (DBPC) and less than about 50% by weight of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof.

16: The composition of claim 11, wherein the gas comprises a perfluorocarbon gas.

17. (canceled)

18: The composition of claim 11, wherein the membrane of each nanobubble consists essentially of dibehenoylglycerophosphocoline (DBPC), dipalmitoylphosphatidic acid (DPPA), dipalmitoylphosphatidylethanolamine (DPPE), and PEG functionalized distearoylphosphatidylethanolamine (DSPE), propylene glycol, and glycerol.

19: The composition of claim 1, the nanobubbles having an average diameter of about 30 nm to about 600 nm.

20: The composition of claim 11, further comprising at least one targeting moiety that is linked to the membrane of each nanobubble and/or microbubble.

21. (canceled)

22: A method comprising:
providing an adoptive immunotherapy composition as recited in claim 1;
administering the adoptive immunotherapy composition to a subject; and
generating at least one image of a region of interest (ROI) of the subject by ultrasound contrast imaging immune cells of the adoptive immunotherapy composition in the ROI of the subject.

23: The method of claim 22, wherein subject has cancer and the ROI of interest includes cancerous cells or tissue in the subject.

24: The method of claim 22, wherein ultrasound contrast imaging includes applying ultrasound energy to the ROI at a duty cycle of about 1% to about 100%, an ultrasound frequency of about 0.2 kHz to about 50 MHz, an intensity of about 0.1 W/cm² to about 5 W/cm², a pressure amplitude of about 50 kPa to about 10 MPa, and a time of about 1 minute to about 30 minutes.

25: The method of claim 22, wherein the adoptive immunotherapy composition administered to the subject includes at least about 1 million immune cells.

26-52. (canceled)
